(19) United States
(12) Patent Application Publication
(10) Pub. No.: US 2022/0398435 A1
(43) Pub. Date: Dec. 15, 2022

(54) Genetic Testing Method, Model Training Method, Apparatus, Device, and System

(71) Applicant: Alibaba Singapore Holding Private Limited, Singapore (SG)

(72) Inventors: Han Yang, Hangzhou (CN); Fei GU, Hangzhou (CN)

(21) Appl. No.: 17/832,474

(22) Filed: Jun. 3, 2022

(30) Foreign Application Priority Data
Jun. 10, 2021 (CN) 202110649698X

(51) Int. Cl. G06N 3/04; G16B 20/20; G06N 3/08; G16B 40/00

(52) U.S. Cl. CPC G06N 3/0454; G16B 20/20; G06N 3/08; G16B 40/00

(57) ABSTRACT

Methods, apparatuses, devices and systems for genetic testing and model training are provided. A genetic testing method includes: obtaining genetic data to be processed, an average number of genetic segments corresponding to each position in the genetic data to be processed being less than or equal to a preset threshold; inputting the genetic data to be processed into a feature generation network layer for performing a feature extraction operation to obtain genetic features corresponding to the genetic data to be processed and enhanced features corresponding to the genetic features; and inputting the genetic data to be processed and the enhanced features into a genetic identification network layer for performing a genetic testing operation to obtain a testing result. The present disclosure realizes performing feature extraction operations through low-depth genetic data, obtaining genetic features and enhanced features corresponding to the genetic features, and performing testing operations based on the enhanced features.

### CROSS REFERENCE TO RELATED PATENT APPLICATIONS

This application claims priority to Chinese Patent Application No. 202110649698.X, filed on 10 Jun. 2021 and entitled “Genetic Testing Method, Model Training Method, Apparatus, Device, and System,” which is hereby incorporated by reference in its entirety.

### TECHNICAL FIELD

The present disclosure relates to the technical field of gene processing, in particular to genetic testing methods, model training methods, apparatuses, devices, and systems.

### BACKGROUND

Gene sequencing is a novel genetic testing technology, which can analyze and determine a complete sequence of genes from blood or saliva, and predict the possibility of suffering from various diseases, and features and reasonableness of behaviors of individuals. The gene sequencing technology can lock a personal lesion gene so as to perform precaution and treatment of the personal lesion gene in advance.

A gene sequence is composed of a plurality of reads segments, each reads segment is a DNA segment with a specific length. This specific length depends on a reading length of a sequencer, and information in each read segment can include: base sequences, mass sequences, positive and negative strands, etc., wherein the base sequences and the mass sequences correspond to each other one by one. For humans, a reads segment covers 23 pairs of chromosomes, amounting to over 30 hundred million base pairs.

Generally, for humans, a few ten thousand dollars are required for one-time complete genome sequencing. Although the cost of gene sequencing is reduced to some extent with the continuous development of sequencing technology in recent years, this is still not a small expense. Therefore, how to reduce the cost of genetic testing is an urgent problem to be solved.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter. The term “techniques,” for instance, may refer to device(s), system(s), method(s) and/or processor-readable/computer readable instructions as permitted by the context above and throughout the present disclosure.

Embodiments of the present disclosure provide a genetic testing method, a model training method, an apparatus, a device, and a system, which can perform learning and training based on a low-depth genetic sample, a genetic feature corresponding to the genetic sample, and an enhanced feature corresponding to the genetic feature, thereby obtaining a genetic testing model. The genetic testing model so generated can perform a testing operation based on low-depth genetic data, which is beneficial to reducing data processing resources and costs required for genetic testing.

In a first aspect, the embodiments of the present disclosure provide a genetic testing method, which includes:

obtaining genetic data to be processed, wherein an average number of genetic segments corresponding to each position in the genetic data to be processed is less than or equal to a preset threshold;

inputting the genetic data to be processed into a feature generation network layer for performing a feature extraction operation to obtain genetic features corresponding to the genetic data to be processed and enhanced features corresponding to the genetic features; and

inputting the genetic data to be processed and the enhanced features into a genetic identification network layer for performing a genetic testing operation to obtain a testing result.

In a second aspect, the embodiments of the present disclosure provide a genetic testing apparatus, which includes:

a first acquisition module configured to obtain genetic data to be processed, wherein an average number of genetic segments corresponding to each position in the genetic data to be processed is less than or equal to a preset threshold;

a first extraction module configured to input the genetic data to be processed into a feature generation network layer for performing a feature extraction operation, and obtain genetic features corresponding to the genetic data to be processed and enhanced features corresponding to the genetic features; and

a first testing module configured to input the genetic data to be processed and the enhanced features into a genetic identification network layer for performing a genetic testing operation to obtain a testing result.

In a third aspect, the embodiments of the present disclosure provide an electronic device, which includes: a memory and a processor, wherein the memory is configured to store one or more computer instructions, and the one or more computer instructions, when executed by the processor, implement the genetic testing method according to the first aspect.

In a fourth aspect, the embodiments of the present disclosure provides a computer storage medium configured to store a computer program that, when executed by a computer, implements the genetic testing method according to the first aspect.

In a fifth aspect, the embodiments of the present disclosure provide a model training method, which includes:

obtaining a genetic sample, wherein the genetic sample corresponds to a sample mutation result, and an average number of genetic segments corresponding to each position in the genetic sample is less than or equal to a preset threshold;

determining genetic features corresponding to the genetic sample and enhanced features corresponding to the genetic features; and

performing learning and training based on a reference genetic result, the genetic features and the enhanced features corresponding to the genetic sample to obtain a genetic testing model, wherein the genetic testing model is used for performing a feature extraction operation on genetic data and performing a testing operation on the genetic data based on extracted features.

In a sixth aspect, the embodiments of the present disclosure provide a model training apparatus, which includes:

a second acquisition module configured to obtain a genetic sample, wherein the genetic sample corresponds to a sample mutation result, and an average number of genetic segments corresponding to each position in the genetic sample is less than or equal to a preset threshold;

a second determination module configured to determine genetic features corresponding to the genetic sample and enhanced features corresponding to the genetic features; and

a second processing module configured to perform learning and training based on a reference genetic result, the genetic features and the enhanced features corresponding to the genetic sample to obtain a genetic testing model, wherein the genetic testing model is used for performing a feature extraction operation on genetic data and performing a testing operation on the genetic data based on extracted features.

In a seventh aspect, the embodiments of the present disclosure provide an electronic device, which includes: a memory and a processor, wherein the memory is configured to store one or more computer instructions, and the one or more computer instructions, when executed by the processor, implement the model training method according to the fifth aspect.

In an eighth aspect, the embodiments of the present disclosure provides a computer storage medium configured to store a computer program that, when executed by a computer, implements the model training method according to the fifth aspect.

In a ninth aspect, the embodiments of the present disclosure provide a genetic testing method, which includes:

obtaining genetic data to be processed, wherein an average number of genetic segments corresponding to each position in the genetic data to be processed is less than or equal to a preset threshold;

determining a genetic testing model for analyzing and processing the genetic data to be processed, wherein the genetic testing model is trained to perform a feature extraction operation on the genetic data to be processed and perform a testing operation on the genetic data to be processed based on extracted features; and

analyzing and processing the genetic data to be processed using the genetic testing model to obtain a testing result.

In a tenth aspect, the embodiments of the present disclosure provide a genetic testing apparatus, which includes:

a third acquisition module configured to obtain genetic data to be processed, wherein an average number of genetic segments corresponding to each position in the genetic data to be processed is less than or equal to a preset threshold;

a third determination module configured to determine a genetic testing model for analyzing and processing the genetic data to be processed, wherein the genetic testing model is trained to perform a feature extraction operation on the genetic data to be processed and perform a testing operation on the genetic data to be processed based on extracted features; and

a third processing module configured to analyze and process the genetic data to be processed using the genetic testing model to obtain a testing result.

In an eleventh aspect, the embodiments of the present disclosure provide an electronic device, which includes: a memory and a processor, wherein the memory is configured to store one or more computer instructions, and the one or more computer instructions, when executed by the processor, implement the genetic testing method according to the ninth aspect.

In a twelfth aspect, the embodiments of the present disclosure provides a computer storage medium configured to store a computer program that, when executed by a computer, implements the genetic testing method according to the ninth aspect.

In a thirteenth aspect, the embodiments of the present disclosure provide a model training method, which includes:

determining a processing resource corresponding to a model training service in response to a request for calling model training; and

performing the following steps with the processing resource: obtaining a genetic sample, wherein the genetic sample corresponds to a sample mutation result, and an average number of genetic segments corresponding to each position in the genetic sample is less than or equal to a preset threshold; determining genetic features corresponding to the genetic sample and enhanced features corresponding to the genetic features; and performing learning and training based on a reference genetic result, the genetic features and the enhanced features corresponding to the genetic sample to obtain a genetic testing model, wherein the genetic testing model is used for performing a feature extraction operation on genetic data and performing a testing operation on the genetic data based on extracted features.

In a fourteenth aspect, the embodiments of the present disclosure provide a model training apparatus, which includes:

a fourth determination module configured to determine a processing resource corresponding to a model training service in response to a request for calling model training; and

a fourth processing module configured to perform the following steps using the processing resource: obtaining a genetic sample, wherein the genetic sample corresponds to a sample mutation result, and an average number of genetic segments corresponding to each position in the genetic sample is less than or equal to a preset threshold; determining genetic features corresponding to the genetic sample and enhanced features corresponding to the genetic features; and performing learning and training based on a reference genetic result, the genetic features and the enhanced features corresponding to the genetic sample to obtain a genetic testing model, wherein the genetic testing model is used for performing a feature extraction operation on genetic data and performing a testing operation on the genetic data based on extracted features.

In a fifteenth aspect, the embodiments of the present disclosure provide an electronic device, which includes: a memory and a processor, wherein the memory is configured to store one or more computer instructions, and the one or more computer instructions, when executed by the processor, implement the model training method according to the thirteenth aspect.

In a sixteenth aspect, the embodiments of the present disclosure provide a computer storage medium configured to store a computer program that, when executed by a computer, implements the model training method according to the thirteenth aspect.

In a seventeenth aspect, the embodiments of the present disclosure provide a genetic testing method, which includes:

determining a processing resource corresponding to a model training service in response to a request for calling model training; and

performing the following steps using the processing resource: obtaining genetic data to be processed, wherein an average number of genetic segments corresponding to each position in the genetic data to be processed is less than or equal to a preset threshold; determining a genetic testing model for analyzing and processing the genetic data to be processed, wherein the genetic testing model is trained to perform a feature extraction operation on the genetic data to be processed and perform a testing operation on the genetic data to be processed based on extracted features; and analyzing and processing the genetic data to be processed using the genetic testing model to obtain a testing result.

In an eighteenth aspect, the embodiments of the present disclosure provide a genetic testing apparatus, which includes:

a fifth determination module configured to determine a processing resource corresponding to a model training service in response to a request for calling model training; and

a fifth processing module configured to perform the following steps using the processing resource: obtaining genetic data to be processed, wherein an average number of genetic segments corresponding to each position in the genetic data to be processed is less than or equal to a preset threshold; determining a genetic testing model for analyzing and processing the genetic data to be processed, wherein the genetic testing model is trained to perform a feature extraction operation on the genetic data to be processed and perform a testing operation on the genetic data to be processed based on extracted features; and analyzing and processing the genetic data to be processed using the genetic testing model to obtain a testing result.

In a nineteenth aspect, the embodiments of the present disclosure provide an electronic device, which includes: a memory and a processor, wherein the memory is configured to store one or more computer instructions, and the one or more computer instructions, when executed by the processor, implement the genetic testing method according to the seventeenth aspect.

In a twentieth aspect, the embodiments of the present disclosure provide a computer storage medium configured to store a computer program that, when executed by a computer, implements the genetic testing method according to the seventeenth aspect.

In a twenty-first aspect, the embodiments of the present disclosure provide a genetic testing system, which includes:

a gene sequence acquisition end configured to obtain genetic data to be processed to be processed and transmit the genetic data to be processed to a genetic testing end, wherein an average number of genetic segments corresponding to each position in the genetic data to be processed is less than or equal to a preset threshold; and

the genetic testing end in communication connection with the gene sequence acquisition end and configured to determine a genetic testing model for analyzing and processing the genetic data to be processed, wherein the genetic testing model is trained for performing a feature extraction operation on the genetic data to be processed and performing a testing operation on the genetic data to be processed based on extracted features; and analyzing and processing the genetic data to be processed using the genetic testing model to obtain a testing result.

According to the technical solutions provided by the embodiments, a genetic sample is obtained, and genetic features corresponding to the genetic sample and enhanced features corresponding to the genetic features are then determined. Performing a feature extraction operation through low-depth genetic data, obtaining genetic features and the enhanced features corresponding to the genetic features, and performing a testing operation based on the enhanced features are thus realized. This not only ensures the accuracy of a genetic testing result is ensured, but also helps reducing data processing resources and costs required by genetic testing, thereby further improving the practicability of the genetic testing method.

### BRIEF DESCRIPTION OF THE DRAWINGS

In order to more clearly illustrate the technical solutions in the embodiments of the present disclosure, drawings that are used for describing the embodiments or the existing technologies will be briefly described below. Apparently, the drawings in the following description represent some embodiments of the present disclosure. One skilled in the art can also obtain other drawings according to the drawings without making any creative effort.

FIG. 1 is a schematic diagram of a scenario of a genetic testing method according to the embodiments of the present disclosure.

FIG. 2 is a schematic diagram of a scenario of a model training method according to the embodiments of the present disclosure.

FIG. 3 is a schematic flowchart of a genetic testing method according to the embodiments of the present disclosure.

FIG. 4 is a schematic flowchart of a model training method according to the embodiments of the present disclosure.

FIG. 5 is a schematic flowchart of a genetic testing model obtained by performing learning training based on a reference genetic result, genetic features and enhanced features corresponding to a genetic sample according to the embodiments of the present disclosure.

FIG. 6 is a schematic flowchart of another model training method according to the embodiments of the present disclosure.

FIG. 7 is a schematic diagram of a first type of genetic sample without mutation according to the embodiments of the present disclosure.

FIG. 8 is a first schematic diagram of a second type of genetic sample with mutation according to the embodiments of the present disclosure.

FIG. 9 is a second schematic diagram of a second type of genetic sample with mutation according to the embodiments of the present disclosure.

FIG. 10 is a third schematic diagram of a second type of genetic sample with mutation according to the embodiments of the present disclosure.

FIG. 11 is a schematic flowchart of another model training method according to the embodiments of the present disclosure.

FIG. 12 is a schematic flowchart of another model training method according to the embodiments of the present disclosure.

FIG. 13 is a schematic flowchart of another model training method according to the embodiments of the present disclosure.

FIG. 14 is a schematic flowchart of a genetic testing method according to the embodiments of the present disclosure.

FIG. 15 is a schematic flowchart of a genetic testing method according to application embodiments of the present disclosure.

FIG. 16 is a schematic flowchart of another model training method according to the embodiments of the present disclosure.

FIG. 17 is a schematic flowchart showing another genetic testing method according to the embodiments of the present disclosure.

FIG. 18 is a schematic structural diagram of a genetic testing apparatus according to the embodiments of the present disclosure.

FIG. 19 is a schematic structural diagram of an electronic device corresponding to the genetic testing apparatus provided in the embodiments shown in FIG. 18.

FIG. 20 is a schematic structural diagram of a model training apparatus according to the embodiments of the present disclosure.

FIG. 21 is a schematic structural diagram of an electronic device corresponding to the model training apparatus provided in the embodiments shown in FIG. 20.

FIG. 22 is a schematic structural diagram of a genetic testing apparatus according to the embodiments of the present disclosure.

FIG. 23 is a schematic structural diagram of an electronic device corresponding to the genetic testing apparatus provided in the embodiments shown in FIG. 22;

FIG. 24 is a schematic structural diagram of another model training apparatus according to the embodiments of the present disclosure.

FIG. 25 is a schematic structural diagram of an electronic device corresponding to the model training apparatus provided in the embodiments shown in FIG. 24.

FIG. 26 is a schematic structural diagram of another genetic testing apparatus according to the embodiments of the present disclosure.

FIG. 27 is a schematic structural diagram of an electronic device corresponding to the genetic testing apparatus provided in the embodiments shown in FIG. 26.

FIG. 28 is a schematic structural diagram of a genetic testing system according to the embodiments of the present disclosure.

### DETAILED DESCRIPTION

In order to make the objectives, technical solutions and advantages of the embodiments of the present disclosure clearer, the technical solutions in the embodiments of the present disclosure will be clearly and completely described below with reference to the drawings in the embodiments of the present disclosure. Apparently, the described embodiments represent some, but not all, of the embodiments of the present disclosure. All other embodiments, which can be derived by one skilled in the art from the embodiments given herein without making any creative effort, shall fall within the scope of protection of the present disclosure.

The terminology used in the embodiments of the present disclosure is intended to describe particular embodiments only, and is not intended to limit the present disclosure. As used in the examples of the present disclosure and the appended claims, singular forms “a”, “an”, and “the” are intended to include plural forms as well, and “a” and “an” generally include at least two, but do not exclude at least one, unless the context clearly dictates otherwise.

It should be understood that the term “and/or” as used herein is merely a type of association that describes associated objects, which means that three relationships may exist. For example, A and/or B may mean: three situations, namely, A exists alone, A and B exist simultaneously, and B exists alone. In addition, the character “/” herein generally indicates that the former and latter related objects are in an “or” relationship.

The word “if”, as used herein, may be interpreted as “at the time when . . . ” or “when . . . ” or “in response to determining” or “in response to testing”, depending on the context. Similarly, the phrases “if determining” or “if testing (a stated condition or event)” may be interpreted as “when determining” or “in response to determining” or “when testing (a stated condition or event)” or “in response to testing (a stated condition or event)”, depending on the context.

It is also noted that the terms “including”, “containing” or any other variation thereof, are intended to cover a non-exclusive inclusion, such that a product or system that includes a list of elements does not include only those elements, but may also include other elements that are not expressly listed or that are inherent to such product or system. Without further limitation, an element defined by the phrase “including a . . . ” does not exclude the presence of other identical elements in a product or system that includes such element.

In addition, a time sequence of steps in each method embodiment described below is only an example and is not strictly limited.

### Definition of Terms

Gene sequencing: is a novel genetic testing technology, can analyze and determine the complete sequence of genes from blood or saliva, and can predict the possibility of suffering from various diseases and the behavior features and behavior reasonableness of individuals. The gene sequencing technology can lock a personal lesion gene so as to perform precaution and treatment based on the personal lesion gene in advance.

Mutation analysis: genetic variation refers to a sudden heritable variation that occurs in a genomic DNA molecule. At the molecular level, genetic variation refers to a structural change in base pair composition or arrangement of genes. Although being relatively stable, genes are able to replicate themselves precisely at cell division. However, such stability is relative. Under some conditions, a gene can also be suddenly changed from its original form to a new form. In short, a new gene is suddenly appeared at a site to replace the original gene.

SNP: single nucleotide polymorphism refers to DNA sequence polymorphism caused by variation of a single nucleotide at the genomic level. It is the most common one of human heritable variations, accounting for over 90% of all known polymorphisms. SNP is widely present in the human genome, averaging 1 per 300 base pairs, and the total number is estimated to be 3 million or even more. A SNP is a two-state marker, caused by a transition or transversion of a single base, or by an insertion or deletion of a base. SNP may be in either a gene sequence or a non-coding sequence outside a gene.

Indel: insertion-deletion, which is as translated as an indel marker, refers to a difference between two parents in the entire genomes. One parent has a certain number of nucleotide insertions or deletions in its genome relative to the other parent. Based on insertion and deletion sites in the genome, primers for Polymerase Chain Reaction (PCR) for amplifying the insertion and deletion sites are designed, which are InDel markers.

Reads: refers to a piece of DNA of a specific length, which is determined by the reading length of a sequencer.

Deep learning: is to lean intrinsic rules and expression levels of sample data, and information obtained in these learning processes is very helpful for interpreting data such as characters, images and sounds, etc. The final goal thereof is to enable a machine to possess the analysis and learning capability like a human, and to recognize data such as characters, images and sounds, etc.

Convolutional Neural Networks (abbreviated as CNN): are a type of feedforward neural networks including convolutional computations and having a deep structure, and are one of the representative algorithms for deep learning.

Generative Adversarial Networks (abbreviated as GAN): are a type of deep learning models and are one of the methods which have prospect in non-supervised learning on complex distribution in recent years. The model uses mutual game learning of (at least) two modules (Generative Model and Discriminative Model) in the framework to yield a reasonably good output.

Sequencing depth refers to an average number of times that a single base on a genome that is tested is sequenced. For example, the sequencing depth of a certain sample is 30×, that is, each single base on the genome of that sample is sequenced (or read) for 30 times on average. Apparently, there are maximum and minimum values of sequencing depth, which are obtained by information analysis. In fact, in order to improve the accuracy, the sequencing depth is generally 15×.

In order to understand specific processes of implementation of the technical solutions in the embodiments of the present disclosure, related technologies are described as follows:

For humans, a Reads segment covers 23 pairs of chromosomes, amounting to more than 30 hundred million base pairs. Information in each read segment may include: base sequences, mass sequences, positive and negative strands, etc., wherein the base sequences and the mass sequences correspond to each other one by one. In this case, how to effectively utilize these enormous pieces of sequencing information and test mutation sites and related properties of mutations is a challenging task.

Generally, for humans, a few ten thousand dollars are required for one-time complete genome sequencing. Although the cost of gene sequencing is reduced to some extent with the continuous development of sequencing technology in recent years, this is still not a small expense. Therefore, how to reduce the cost of genetic testing is an urgent problem to be solved.

Since the price of sequencing is strictly and positively correlated with the depth of sequencing data, the cost will be greatly reduced if highly accurate variant identification can still be achieved for a low-depth sequencing result from the perspective of sequencing depth. For example, if the accuracy of a mutation analysis algorithm on data of depth of 20 times can be made to be close to that of depth of 40 times, the sequencing cost can then be reduced by a factor of two.

At present, a way of implementing a genetic mutation testing method includes: obtaining genetic data, determining low-depth data features corresponding to the genetic data, converting the low-depth data features into high-depth data features using a conversion model, and inputting the high-depth data features into a variant identification model for performing analysis and processing, so that a variant identification result can be obtained.

Although the above method can obtain a relatively accurate variant identification result, the above method has the following problems: the conversion model and the variant identification model are not trained end to end, so that an optimization mode of the conversion model and the variant identification model is relatively complex, and the quality and efficiency of optimization of methods of testing genetic mutation are reduced.

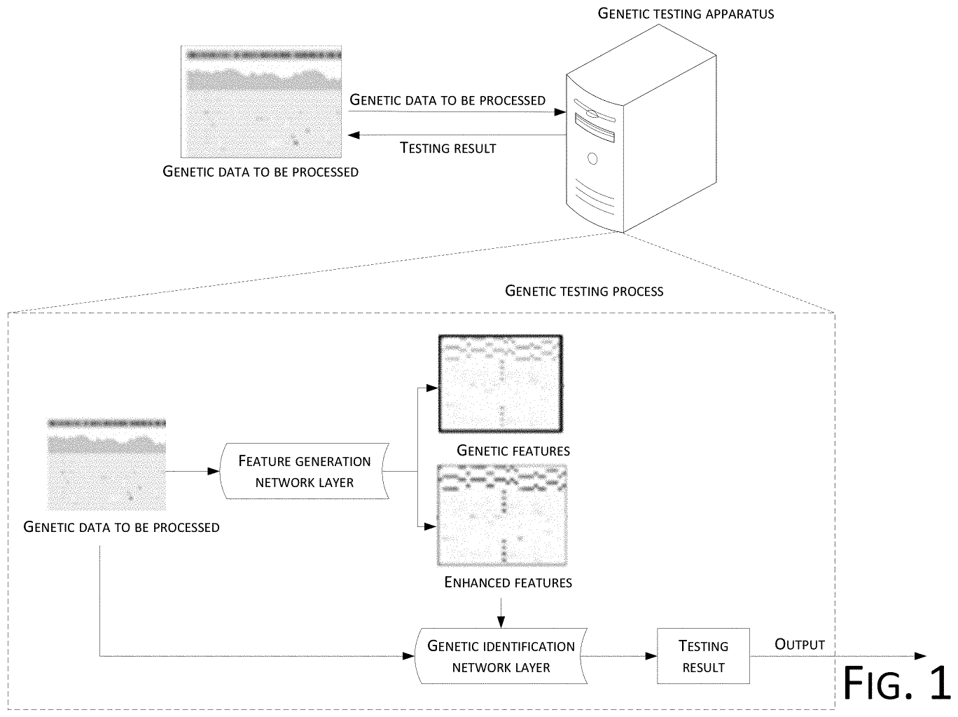

In order to solve the above technical problems, the embodiments of the present disclosure provide genetic testing methods, model training methods, apparatuses and devices, wherein an execution subject of the genetic testing methods may be a genetic testing apparatus. The genetic testing apparatus may be provided with a preset interface, and genetic data to be processed may be transmitted to the genetic testing apparatus through the preset interface, so that a genetic testing model may perform a genetic testing operation on the genetic data to be processed, which can be referenced to those shown in FIG. 1.

The genetic testing apparatus may be a device capable of providing a genetic testing service in a network virtual environment, and generally refers to a device that performs information processing and genetic testing operations using a network. In physical implementations, the genetic testing apparatus can be any device capable of providing computing services, responding to service requests, and performing processing, and can be, for example, a cluster server, a conventional server, a cloud server, a cloud host, a virtual center, and the like. The genetic testing apparatus mainly includes a processor, a hard disk, a memory, a system bus and the like, and is similar to a general computer framework.

In the above embodiments, genetic data to be processed may be stored in a set device, and the set device may perform a network connection with the genetic testing apparatus to obtain the genetic data to be processed, where the network connection may be a wireless or wired network connection. If the set device is in communication connection with the genetic testing apparatus, a network format of such mobile network may be any one of 2G GSM), 2.5G (GPRS), 3G (WCDMA, TD-SCDMA, CDMA2000, UTMS), 4G (LTE), 4G+(LTE+), WiMax, 5G, and the like.

The genetic testing apparatus is used for receiving genetic data to be processed for performing a genetic testing operation, and inputting the genetic data to be processed to a feature generation network layer for performing a feature extraction operation to obtain genetic features corresponding to the genetic data to be processed and enhanced features corresponding to the genetic features, wherein an average number of genetic segments corresponding to each position in the genetic data to be processed is less than or equal to a preset threshold; inputting the genetic data to be processed and the enhanced features into a genetic identification network layer for performing a genetic testing operation to obtain a testing result. As such, a feature extraction operation is performed on low-depth genetic data to obtain genetic features and enhanced features corresponding to the genetic features, and a testing operation is performed based on the enhanced features. This not only ensures the accuracy of a genetic testing result, but also reduces the cost and amount of data processing.

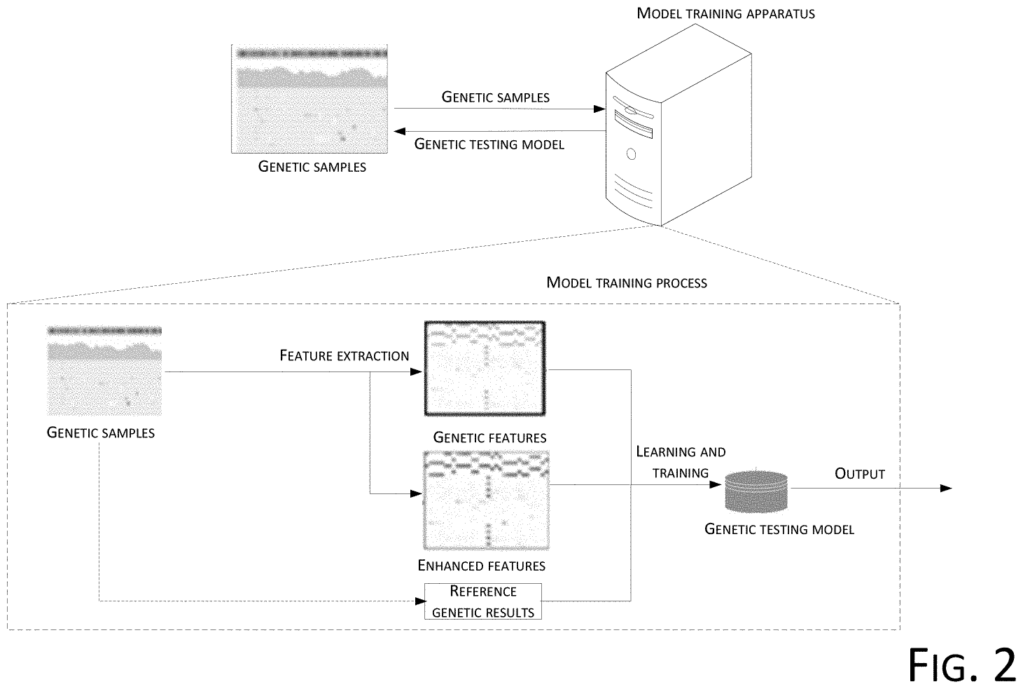

In addition, an execution subject of the above model training method may be a model training apparatus. The model training apparatus may be provided with a preset interface, and a genetic sample may be transmitted to the model training apparatus through the preset interface, so that the model training apparatus may perform a model training operation based on the obtained genetic sample, specifically, as shown in FIG. 2.

The model training apparatus may be a device capable of providing a genetic testing service in a network virtual environment, and generally refers to a device that performs information processing and genetic testing operations using a network. In physical implementations, the genetic testing apparatus can be any device capable of providing computing services, responding to service requests, and performing processing, and can be, for example, a cluster server, a conventional server, a cloud server, a cloud host, a virtual center, and the like. The genetic testing apparatus mainly includes a processor, a hard disk, a memory, a system bus and the like, and is similar to a general computer framework.

In the above embodiments, the genetic sample may be stored in a set device, and the set device may perform a network connection with the model training apparatus to obtain the genetic sample, where the network connection may be a wireless or wired network connection. If the set device and the model training apparatus are in communication connection, a network format of such mobile network may be any one of 2G (GSM), 2.5G (GPRS), 3G (WCDMA, TD-SCDMA, CDMA2000, UTMS), 4G (LTE), 4G+(LTE+), WiMax, 5G, and the like.

The model training apparatus is configured to receive a genetic sample for performing a model training operation, wherein the genetic sample corresponds to a sample mutation result, an average number of genetic segments corresponding to each position in the genetic sample is less than or equal to a preset threshold, i.e., the genetic sample is low-depth sample data. After the genetic sample is obtained, a feature extraction operation can be performed on the genetic sample, so that genetic features corresponding to the genetic sample and enhanced features corresponding to the genetic features can be obtained, then learning and training can be performed based on a reference genetic result, the genetic features and the enhanced features corresponding to the genetic sample, and a genetic testing model capable of implementing a genetic testing operation can be obtained.

According to the technical solutions provided by the embodiments, a genetic sample is obtained, a feature extraction operation is performed on the genetic sample, so that genetic features corresponding to the genetic sample and enhanced features corresponding to the genetic features can be determined, learning and training can be effectively performed based on low-depth genetic sample, genetic features corresponding to the genetic sample and enhanced features corresponding to the genetic features. A genetic testing model can thereby be obtained, and the genetic testing model so generated can perform a testing operation based on low-depth genetic data. This effectively reduces resources and cost of data processing required by genetic testing, thus further improving the practicability of such model training method.

Some embodiments of the present disclosure are described in detail below with reference to accompanying drawings. Whenever there is no conflict between embodiments, the embodiments and features of the embodiments described below may be combined with each other.

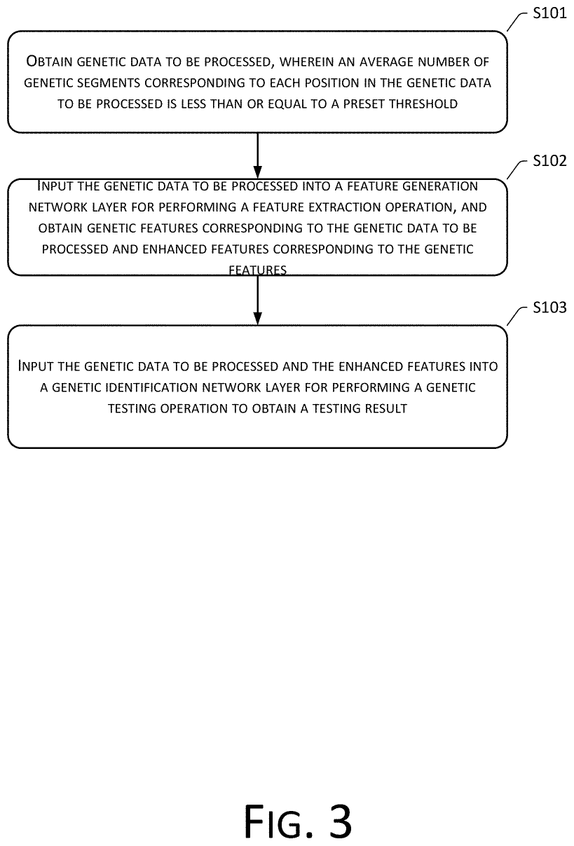

FIG. 3 is a schematic flowchart of a genetic testing method according to the embodiments of the present disclosure. Referring to FIG. 3, the embodiments provide a genetic testing method. An execution subject of the method can be a genetic testing apparatus. It can be understood that the genetic testing apparatus can be implemented as software, or a combination of software and hardware. Specifically, the genetic testing method can include the following steps:

Step S101: Obtain genetic data to be processed, wherein an average number of genetic segments corresponding to each position in the genetic data to be processed is less than or equal to a preset threshold.

Step S102: Input the genetic data to be processed into a feature generation network layer for performing a feature extraction operation, and obtain genetic features corresponding to the genetic data to be processed and enhanced features corresponding to the genetic features.

Step S103: Input the genetic data to be processed and the enhanced features into a genetic identification network layer for performing a genetic testing operation to obtain a testing result.

The above steps are explained in detail below:

Step S101: Obtain genetic data to be processed, wherein an average number of genetic segments corresponding to each position in the genetic data to be processed is less than or equal to a preset threshold.

The genetic data to be processed refers to genetic data that needs to be subjected to a genetic testing operation. The genetic testing operation may include a genetic feature testing operation. The genetic feature testing operation may include: a gene stability testing, a gene variability testing operation (i.e., a genetic mutation testing operation), and the like. Specifically, a technical person in the embodiments of the present disclosure can perform configuration of the genetic testing operation according to a specific application scenario or application requirements, and details thereof are not repeated herein. In addition, each position in the genetic data to be processed may correspond to a plurality of genetic segments, and the genetic segments may include qualit(ies) of base(s). It is understood that the genetic segments may include not only the above qualit(ies) of base(s), but also other information. For example, a genetic segment may include information such as base information (A, C, G, T), mapping qualit(ies), positive and negative strands (A, C, G, T, A-, C-, G-, T-, wherein the latter four strands are negative strands and the former four strands are positive strands), etc.

It needs to be noted that the average number of genetic segments corresponding to each position in the genetic data to be processed is less than or equal to a preset threshold, that is, the genetic data to be processed is limited to genetic data with a low depth. It is understood that the preset threshold is an upper limit configured in advance for limiting genetic data with a low depth, and a specific numerical range thereof may be adjusted based on different application scenarios or application requirements. For example, the preset threshold may be 10×, 15×, or 20×, etc. For example, when the preset threshold is 15× and the average number of genetic segments corresponding to each position in the genetic data to be processed is less than or equal to 15×, this indicates that the genetic data to be processed is low-depth genetic data. When the average number of genetic segments corresponding to each position in the genetic data to be processed is more than 15×, this indicates that the genetic data to be processed is high-depth genetic data. In order to reduce the cost required by genetic testing, genetic data to be processed whose average number of genetic segments corresponding to each position is less than or equal to a preset threshold is obtained from the sequence, so that a genetic testing operation based on the low-depth genetic data to be processed can be realized.

In addition, the embodiments do not limit specific methods of obtaining the genetic data to be processed. For example, the genetic data to be processed may be stored in a set region, and the genetic data to be processed may be obtained by accessing the set region. In other examples, a genetic testing apparatus may be provided with a gene collection module, and obtain pending genetic data through the gene collection module. In different application scenarios, the gene collection module can correspondingly have different structural features. For example, in obtaining genetic data to be processed through blood, the gene collection module may be a blood collector. Specifically, the blood testor collects blood from a body of a set object (a person, an animal, or the like) and extracts the genetic data to be processed from the blood. Similarly, when the genetic data to be processed is obtained through saliva, the gene collection module may be a saliva collector. Specifically, the saliva testor collects saliva from the body of a set object (a person, an animal, or the like), and the genetic data to be processed is extracted from the saliva. Similarly, when the genetic data to be processed is acquired through skin, the gene collection module may be a skin collector. Specifically, the skin collector obtains the skin from the body of a set object (person, animal, etc.), and extracts the genetic data to be processed from the skin.

Apparently, one skilled in the art may also use other methods to obtain the genetic data to be processed, as long as the accuracy and reliability of obtaining the genetic data to be processed can be ensured, and details thereof are not described herein.

Step S102: Input the genetic data to be processed into a feature generation network layer for performing a feature extraction operation, and obtain genetic features corresponding to the genetic data to be processed and enhanced features corresponding to the genetic features.

A genetic testing model for performing genetic testing operations on genetic data to be processed is trained in advance. The genetic testing model can include: a feature generation network layer and a genetic identification network layer which is in communication connection with the feature generation network layer. The feature generation network layer is used for implementing feature extraction operations, and the genetic identification network layer is used for implementing genetic testing operations. After the genetic data to be processed is obtained, the genetic data to be processed can be inputted into the feature generation network layer, and a feature extraction operation is performed on the genetic data to be processed using the feature generation network layer, so that genetic features corresponding to the genetic data to be processed and enhanced features corresponding to the genetic features can be obtained.

Step S103: Input the genetic data to be processed and the enhanced features into a genetic identification network layer for performing a genetic testing operation to obtain a testing result.

After the enhanced features are obtained, the genetic data to be processed and the enhanced features can be inputted into a genetic identification network layer. The genetic identification network layer can perform a genetic testing operation based on the genetic data to be processed and the enhanced features, so that a testing result can be obtained. In some examples, inputting the genetic data to be processed and the enhanced features into the genetic identification network layer for performing the genetic testing operation to obtain the testing result may include: performing genetic testing processing on the genetic data to be processed and the enhanced features using the genetic identification network layer to obtain testing reference information corresponding to the genetic data to be processed, wherein the testing reference information includes at least one of the following information: 21-type genotype prediction information, zygotic prediction information, first allelic mutation length information, and second allelic mutation length information; and obtaining the testing result corresponding to the genetic data to be processed according to the testing reference information.

After the genetic testing model and the genetic data to be processed are obtained, the genetic testing model may be used to perform testing processing on the genetic data for performing analysis processing, so that mutation reference information corresponding to the genetic data may be obtained. The mutation reference information may include at least one of: 21-type genotype prediction information, zygote prediction information, first allelic mutation length information, and second allelic mutation length information, wherein the 21-type genotypes for which the 21-type genotype prediction information is directed include: ‘AA’, ‘AC’, ‘AG’, ‘AT’, ‘CC’, ‘CG’, ‘CT’, ‘GG’, ‘GT’, ‘TT’, ‘AI’, ‘CI’, ‘GI’, ‘TI’, ‘AD’, ‘CD’, ‘GD’, ‘TD’, ‘II’, and ‘DD’, wherein A, C, G, T is four bases, and I and D are insertion and deletion respectively. The zygotic prediction information includes three types: homozygous and identical to reference base(s), homozygous and inconsistent with the reference base(s), and heterozygous. In the first allelic mutation length information, SNP mutation is 0, and Indel mutation is the length of corresponding insertion(s) and deletion(s). In the length of the second allelic mutation, SNP mutation, and Indel mutation is the length of corresponding insertion(s) and deletion(s).

After obtaining the mutation reference information corresponding to the genetic data, the mutation reference information may be analyzed to obtain a testing result. It is understood that the testing result is obtained based on at least one of the 21-type genotype prediction information, the zygote prediction information, the first allelic mutation length information, and the second allelic mutation length information, thereby ensuring the accuracy and reliability of determining the testing result.

According to the genetic testing method provided by the embodiments of the present disclosure, a genetic sample is obtained, genetic features corresponding to the genetic sample and enhanced features corresponding to the genetic features are then determined. This enables a feature extraction operation to be performed through low-depth genetic data. Genetic features and enhanced features corresponding to the genetic features are obtained, and a testing operation is performed based on the enhanced features. This not only ensures the accuracy of a genetic testing result, but also reduces resources and cost of data processing required by genetic testing, thus further improving the practicability of the genetic testing method.

In some examples, the method in the embodiments of the present disclosure may further include: obtaining a standard data type corresponding to the genetic data to be processed; inputting the genetic features into the data identification network layer to perform a data type identification operation to obtain genetic data types; determining a loss function for a feature generation network layer based on the genetic data types and the standard data type; and optimizing the feature generation network layer using the loss function to obtain an optimized feature generation network layer.

The genetic data to be processed may correspond to attribute information of a standard data type, and the standard data type may include genetic data to be processed in normal state (i.e., genetic data without genetic mutation) and genetic data to be processed in abnormal state (i.e., genetic data with genetic mutation). When performing feature extraction operations on different data types of genetic data to be processed, different feature extraction logics may be provided correspondingly. Therefore, in order to improve the quality and efficiency of genetic testing operations, after obtaining a feature generation network layer, optimization processing can be performed for the feature generation network layer. Specifically, a standard data type corresponding to genetic data to be processed may be obtained first, and genetic features corresponding to the genetic data to be processed are inputted to the data identification network layer to perform a data type identification operation, so as to obtain genetic data type(s). A loss function is then determined based on the obtained genetic data type(s) and the standard data type, and the feature generation network layer is optimized using the loss function to obtain an optimized feature generation network layer.

In some examples, the feature generation network layer includes a portion of the data identification network layer. Optimizing the feature generation network layer using the loss function to obtain the optimized feature generation network layer may include: optimizing the data identification network layer based on the loss function to obtain an optimized data identification network layer; and determining the optimized feature generation network layer based on the optimized data identification network layer.

Since the feature generation network layer includes a part of the data identification network layer, that is, network parameters in the data identification network layer are the same as those in the feature generation network layer. At this time, an optimization operation of the feature generation network layer can be realized by optimizing the data identification network layer. Specifically, genetic data to be processed, genetic features corresponding to the genetic data to be processed, and standard data type(s) corresponding to a genetic sample may be obtained, and the genetic features may then be analyzed and processed using the data identification network layer to obtain genetic data type(s) corresponding to the genetic data to be processed. A loss function for the feature generation network layer may then be determined based on the genetic features, the genetic data type(s), and the standard data type(s). After the loss function is obtained, the data identification network layer can be optimized using the loss function, so that an optimized data identification network layer can be obtained.

In the embodiments of the present disclosure, standard data type(s) corresponding to genetic data to be processed is/are obtained. Genetic features are inputted into a data identification network layer for performing a data type identification operation to obtain genetic data type(s). A loss function for a feature generation network layer is determined based on the genetic data type(s) and the standard data type(s). The loss function is utilized to optimize the feature generation network layer to obtain an optimized feature generation network layer, thus effectively achieving the optimization operation of the feature generation network layer, and further improving the quality and efficiency of feature generation of genetic data by the feature generation network layer.

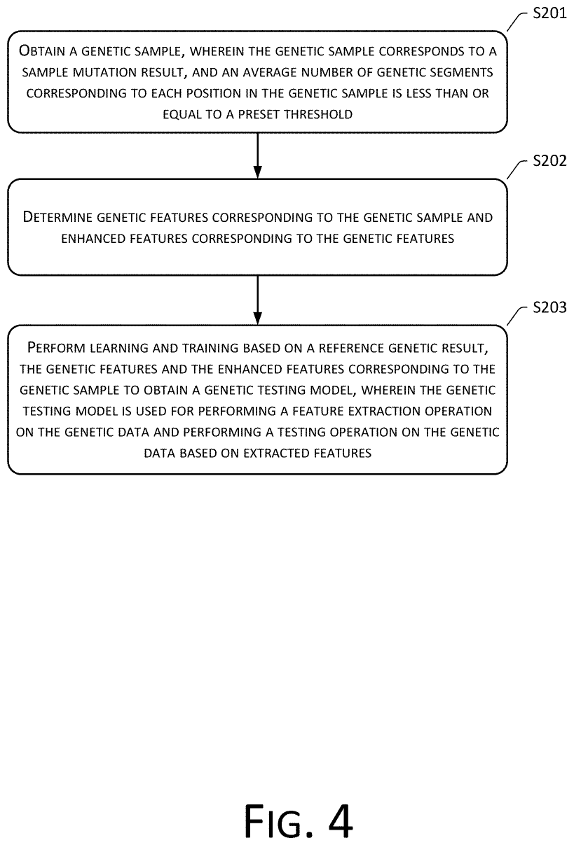

FIG. 4 is a schematic flowchart of a model training method according to the embodiments of the present disclosure. Referring to FIG. 4, the embodiments provide a model training method, and an execution subject of the method may be a model training apparatus. It is understood that the model training apparatus may be implemented as software, or a combination of software and hardware. Specifically, the model training method may include the following steps:

Step S201: Obtain a genetic sample, wherein the genetic sample corresponds to a sample mutation result, and an average number of genetic segments corresponding to each position in the genetic sample is less than or equal to a preset threshold.

Step S202: Determine genetic features corresponding to the genetic sample and enhanced features corresponding to the genetic features.

Step S203: Perform learning and training based on a reference genetic result, the genetic features and the enhanced features corresponding to the genetic sample to obtain a genetic testing model, wherein the genetic testing model is used for performing a feature extraction operation on the genetic data and performing a testing operation on the genetic data based on extracted features.

The above steps are explained in detail below:

Step S201: Obtain a genetic sample, wherein the genetic sample corresponds to a sample mutation result, and an average number of genetic segments corresponding to each position in the genetic sample is less than or equal to a preset threshold.

The genetic samples are sample data which are used for model training operations and correspond to sample mutation results. The number of genetic samples can be one or more. It can be understood that the quality and the effect of model training have a corresponding relationship with the number of genetic samples. When the number of the genetic samples is large, the quality and the effect of data processing of a genetic testing model so trained and generated are also higher, and the time of training of model training operations is correspondingly increased. When the number of genetic samples is small, the quality and the effect of data processing of a genetic testing model so trained and generated are relatively low, and the time of training of model training operations is correspondingly reduced.

Specifically, a genetic sample includes a plurality of base positions, each of which may correspond to a plurality of genetic segments. The genetic segment may include base qualit(ies). It is understood that the genetic segment may include not only the base qualit(ies) as described above, but also other information. For example, the genetic segment may include information such as base information (A, C, G, T), mapping qualit(ies), positive and negative strands (A, C, G, T, A-, C-, G-, T-, wherein the latter four strands are negative strands and the former four strands are positive strands), and the like.

It should be noted that the average number of genetic segments corresponding to each position in the genetic sample is less than or equal to the preset threshold, which means the genetic sample is a low-depth gene sequence. It is understood that the preset threshold is an upper limit configured in advance for defining low-depth genetic samples, and a specific value range thereof may be adjusted based on different application scenarios or application requirements. For example, the preset threshold may be 10×, 15×, or 20×, etc. For example, when the preset threshold is 15× and the average number of genetic segments corresponding to each position in the genetic sample is less than or equal to 15×, this means that the genetic sample is a low-depth genetic sample. When the average number of genetic segments corresponding to each position in the genetic sample is more than 15×, this means that the genetic sample is high-depth genetic data. In order to reduce the cost required by gene sequencing, genetic samples whose average number of genetic segments corresponding to each position in the sequence is less than or equal to a preset threshold are obtained, so that genetic testing operations can be performed based on the genetic samples with low depth.

In addition, the embodiments of the present disclosure are not limited to specific methods of obtaining the genetic sample. For example, the genetic sample may be stored in a set region, and the genetic sample may be obtained by accessing the set region. Alternatively, the genetic sample is stored in a third device, and the third device is in communication connection with the model training apparatus. The model training device is provided with an interactive interface. A user can input an execution operation on the interactive interface, and the model training apparatus can generate a sample acquisition request based on the generated execution operation. The model training apparatus can then obtain the genetic sample from the third device based on the sample acquisition request, and thereby the genetic sample can be stably obtained.

Apparently, one skilled in the art may also use other methods to obtain the genetic sample, as long as the accuracy and reliability of obtaining the genetic sample can be ensured, and details thereof are not repeated herein.

Step S202: Determine genetic features corresponding to the genetic sample and enhanced features corresponding to the genetic features.

After the genetic sample is obtained, the genetic sample can be analyzed to determine genetic features corresponding to the genetic sample and enhanced features corresponding to the genetic features. It needs to be noted that, since the genetic sample is a low-depth genetic sample, genetic features that are obtained by performing a feature extraction operation on the genetic sample are low-depth genetic features, and the amount of information included in the low-depth genetic features is relatively small. Compared to the low-depth genetic features, the data size of the enhanced feature data may be the same as the data size of genetic features, and the enhanced features may include a larger amount of information than the genetic features. Since the amount of information included in the enhanced features is relatively large and the size is the same as the data size of the genetic features, the quality and efficiency of genetic testing operations using a genetic testing model that is generated can be effectively improved when model training is performed based on the enhanced features.

In some examples, determining the genetic features corresponding to the genetic sample may include: obtaining a base quality included in the genetic sample; determining a confidence level corresponding to the genetic sample based on the base quality; and performing the feature extraction operation on the genetic sample based on the confidence level corresponding to the genetic sample to obtain the genetic features.

The genetic sample includes a base quality. After the genetic sample is obtained, an information extraction operation can be performed on the genetic sample, so that the base quality included in the genetic sample can be obtained. Since there is a mapping relationship between the base quality and the confidence level corresponding to the genetic segment, after the base quality included in the genetic sample is obtained, the confidence level corresponding to the genetic sample can be determined based on the base quality included in the genetic sample. In some examples, determining the confidence level corresponding to the genetic sample based on the base quality may include: obtaining ratio information between the base quality and 10; and determining the confidence level corresponding to the genetic sample based on the ratio information, wherein the confidence level is positively correlated with the base quality and is less than 1.

When the base quality (qual) is obtained, the ratio information

$$( \frac{q u a l}{10} )$$

between the base quality (qual) and 10 is obtained. Thereafter, the confidence level (p) corresponding to the genetic sample can be determined based on the ratio information

$$( \frac{q u a l}{10} ) .$$

In some instances, the confidence level is

$$p = 1 - 1 {0}^{- \frac{q u a l}{10}} .$$

At this time, the confidence level (p) is a value between 0 and 1, and the confidence level (p) is positively correlated with the base quality. In other words, the greater the base quality is, the higher the base quality included in the genetic sample is. At this time, the higher the accuracy of the genetic sample is, the confidence level (p) of the genetic segment can also be determined to be higher accordingly. Similarly, the confidence level (p) becomes lower as the base quality becomes lower.

Apparently, one skilled in the art can employ other methods for obtaining confidence levels of genetic samples. For example, the confidence level is

$$p = 1 - 1 {0}^{- \frac{q u a l}{10}} .$$

At this time, the confidence level is negatively correlated with the base quality. In other words, the confidence level (p) is reduced when the base quality is larger, and the confidence level (p) becomes higher as the base quality becomes lower.

Furthermore, after the confidence level corresponding to the genetic sample is obtained, the feature extraction operation may be performed on the genetic sample based on the confidence level corresponding to the genetic sample, so that the genetic features of the genetic sample may be obtained. In some examples, performing the feature extraction operation on the genetic sample based on the confidence level corresponding to the genetic sample, to obtain the genetic features of the genetic sample may include: performing the feature extraction operation on the genetic sample based on the confidence level corresponding to the genetic sample using a statistical counting mode to obtain the genetic features of the genetic sample, wherein the genetic features include: base information, base positions, and statistics corresponding to the base information.

Specifically, the base information may include at least one of: A, G, C, T, A-, G-, C-, and T-, wherein base information (A, G, C, T) is positive strands, and base information (A-, G-, C-, and T-) is negative strands. The statistics corresponding to the base information may include at least one of the following: a statistic of bases being identical to reference bases, a statistic of base insertions, a statistic of base deletions, and a statistic of single nucleotide alternative bases. After the confidence level corresponding to the genetic sample is obtained, the feature extraction operation can be performed on the genetic sample based on the confidence level corresponding to the genetic sample and using a statistical technology, so that the genetic features of the genetic sample can be stably obtained with the help of the confidence level corresponding to the genetic sample, and thereby the completeness and efficiency of extracting the genetic features are improved.

Since the genetic features obtained by performing the feature extraction operation on the genetic sample are low-depth genetic features, the amount of information included in the low-depth genetic features is relatively small. In order to improve the accuracy of model training operations, the genetic features can be enhanced, so that enhanced features corresponding to the genetic features can be obtained, and the amount of information included in the enhanced features obtained thereby is relatively large. In this way, the quality and efficiency of genetic testing operations can be effectively improved when a testing operation is performed based on the enhanced features. In still other examples, determining the enhanced features corresponding to the genetic features may include: obtaining a convolutional neural network model used for enhancing on the genetic features; and enhancing the genetic features based on the convolutional neural network model to obtain the enhanced features corresponding to the genetic features.

Specifically, a convolutional neural network used for enhancing genetic features is configured in advance. The convolutional neural network may be a full convolutional neural network, and the convolutional neural network may be a two-dimensional network model or a three-dimensional network model. Specifically, after the genetic features are obtained, the genetic features may be input into the convolutional neural network model, so that the convolutional neural network model may perform enhancement on the genetic features, and enhanced features corresponding to the genetic features may thus be obtained. The amount of information included in the enhanced features obtained thereby is greater than the amount of information included in the genetic features. The data size of the enhanced features obtained thereby can be the same as the data size of the genetic features, thus facilitating a testing operation based on the enhanced features, and further improving the quality and efficiency of the testing operation.

Step S203: Perform learning and training based on a reference genetic result, the genetic features and the enhanced features corresponding to the genetic sample to obtain a genetic testing model, wherein the genetic testing model is used for performing a feature extraction operation on the genetic data and performing a testing operation on the genetic data based on extracted features.

After the genetic sample is obtained, learning and training may be performed based on a reference genetic result, the genetic features, and the enhanced features corresponding to the genetic sample, so that a genetic testing model may be generated and obtained. The generated genetic testing model is used to perform a feature extraction operation on genetic data, and may perform a testing operation on the genetic data based on extracted features, wherein the testing operation may include a genetic feature testing operation. Specifically, the genetic feature testing operation may include: a gene stability testing, a gene variability testing operation (i.e., a genetic mutation testing operation), etc. A technical person of the embodiments of the present disclosure can perform configuration of the genetic testing operation according to a specific application scenario or application requirement, which is not described herein again.

In the model training method provided by the embodiments of the present disclosure, a genetic sample is obtained, wherein the genetic sample corresponds to a sample mutation result, and an average number of genetic segments corresponding to each position in the genetic sample is less than or equal to a preset threshold. Genetic features corresponding to the genetic sample and enhanced features corresponding to the genetic features are then determined, so that learning and training can be effectively realized based on the genetic samples with low depth, the genetic features corresponding to the genetic samples and the enhanced features corresponding to the genetic features, and a genetic testing model can thereby be obtained. The genetic testing model so generated can perform testing operations based on genetic data with low depth. This not only effectively reduces resources and the cost of data processing required by genetic testing, but also further improves the practicability of the model training method.

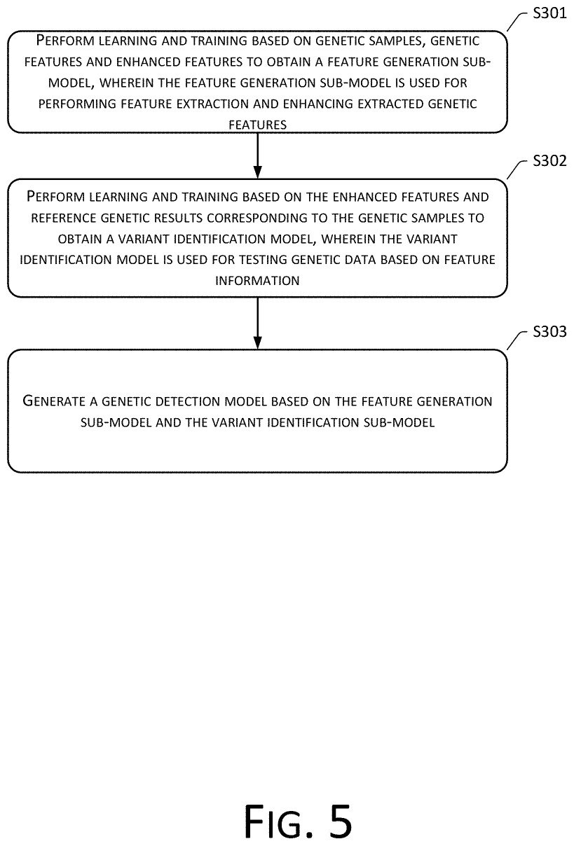

FIG. 5 is a schematic flowchart of a genetic testing model obtained by performing learning and training based on reference genetic results, genetic features and enhanced features corresponding to genetic samples according to the embodiments of the present disclosure. On the basis of the above embodiments, referring to FIG. 5, the embodiments of the present disclosure provide an implementation of performing learning and training based on reference genetic results, genetic features and enhanced features corresponding to genetic samples. Specifically, a genetic testing model to be generated may include: a feature generation sub-model and a variant identification model. At this time, in the embodiments of the present disclosure, performing learning and training based on reference genetic results, genetic features and enhanced features corresponding to genetic samples to obtain the genetic testing model may include:

Step S301: Perform learning and training based on genetic samples, genetic features and enhanced features to obtain a feature generation sub-model, wherein the feature generation sub-model is used for performing feature extraction and enhancing extracted genetic features.

The genetic features are low-depth data features. The enhanced features can be high-depth data features. After genetic samples, genetic features and enhanced features are obtained, association relationships among the genetic samples, the genetic features and the enhanced features can be learned, and therefore a feature generation sub-model can be obtained.

Step S302: Perform learning and training based on the enhanced features and reference genetic results corresponding to the genetic samples to obtain a variant identification model, wherein the variant identification model is used for testing genetic data based on feature information.

After the enhanced features and the reference genetic results corresponding to the genetic samples are obtained, association relationships between the enhanced features and the reference genetic results can be learned and trained, so that a variant identification model can be obtained. The variant identification model can perform testing operations on genetic data based on feature information, and can output testing results corresponding to the genetic data.

Step S303: Generate a genetic testing model based on the feature generation sub-model and the variant identification sub-model.

After the feature generation sub-model and the variant identification model are obtained, a genetic testing model can be generated based on the feature generation sub-model and the variant identification model. The genetic testing model can perform feature extraction operations on genetic data and perform testing operations on the genetic data based on extracted features.

In the embodiments of the present disclosure, a feature generation sub-model is obtained by performing learning and training on genetic samples, genetic features and enhanced features, and a variant identification sub-model is then obtained by performing learning and training on the enhanced features and reference genetic results corresponding to the genetic samples. A genetic testing model can be generated based on the feature generation sub-model and the variant identification sub-model, so that the quality and the effect of learning and training of the genetic testing model are effectively ensured, and the quality and efficiency of testing operations on genetic data based on the genetic testing model are further improved.

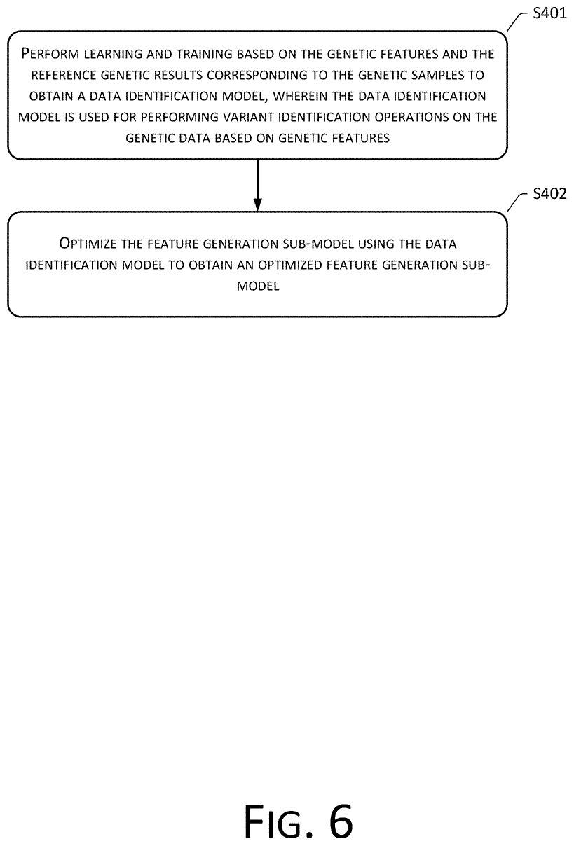

FIG. 6 is a schematic flowchart of another model training method according to the embodiments of the present disclosure. On the basis of the foregoing embodiments, referring to FIG. 6, after obtaining the feature generation sub-model, the method in the embodiments of the present disclosure may further include:

Step S401: Perform learning and training based on the genetic features and the reference genetic results corresponding to the genetic samples to obtain a data identification model, wherein the data identification model is used for performing variant identification operations on the genetic data based on genetic features.

Step S402: Optimize the feature generation sub-model using the data identification model to obtain an optimized feature generation sub-model.

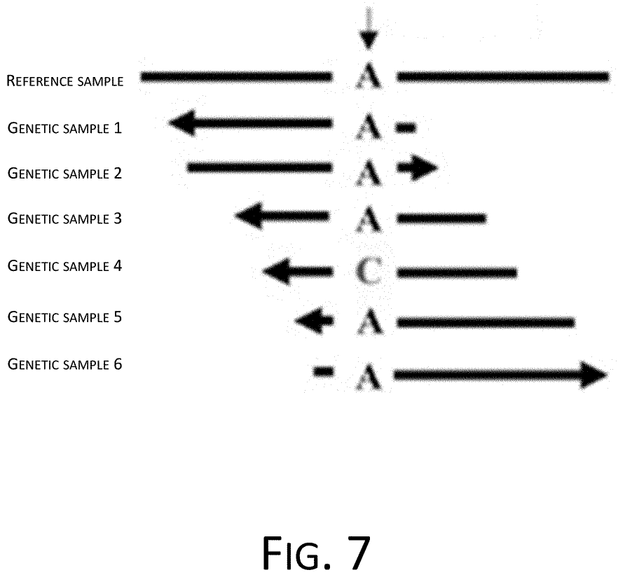

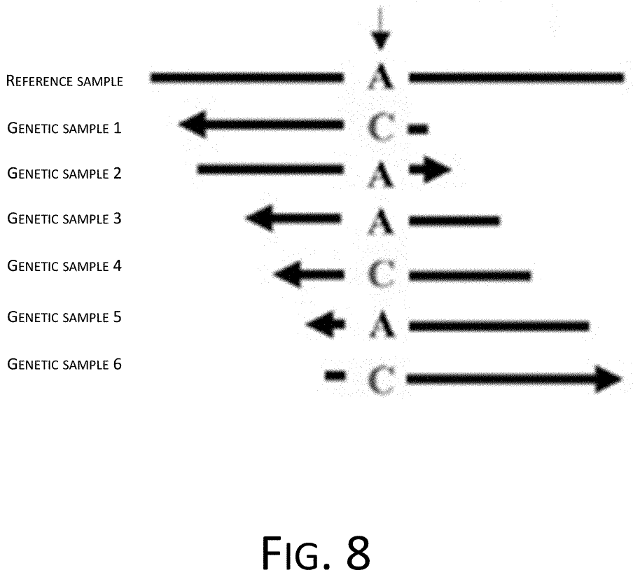

For genetic samples, the genetic samples may include a first type of genetic samples without mutation and a second type of genetic samples with mutation. As shown in FIG. 7, for a base “A” at a certain position of a reference sample, a plurality of genetic samples can be obtained by a plurality of forward testing and reverse testing operations, and the genetic samples are assumed to include: a genetic sample 1, a genetic sample 2, a genetic sample 3, a genetic sample 4, a genetic sample 5, and a genetic sample 6. Although base information “C” at the corresponding position in the genetic sample 4 is different from base information “A” in the reference sample (possibly caused by erroneous testing), base information at the corresponding position in other samples is the same as the base information in the reference sample. In this case, the proportion of the number of samples with different bases obtained by testing is relatively small, and it can thus be considered that the genetic samples obtained by testing have no mutation, and are the first type of genetic samples. On the contrary, referring to FIG. 8, for a base “A” at a certain position of a reference sample, a plurality of genetic samples can be obtained by a plurality of forward testing and reverse testing operations, and the genetic samples are assumed to include: a genetic sample 1, a genetic sample 2, a genetic sample 3, a genetic sample 4, a genetic sample 5, and a genetic sample 6, wherein base information “C” at the corresponding positions in the genetic sample 1, the genetic sample 4 and the genetic sample 6 is different from the base information “A” in the reference sample, while base information at the corresponding positions in other samples is the same as the base information in the reference sample. The proportion of the number of the samples with different bases obtained by testing is relatively large, and it can thus be considered that the genetic samples obtained by testing have mutations and are the second type of genetic samples.

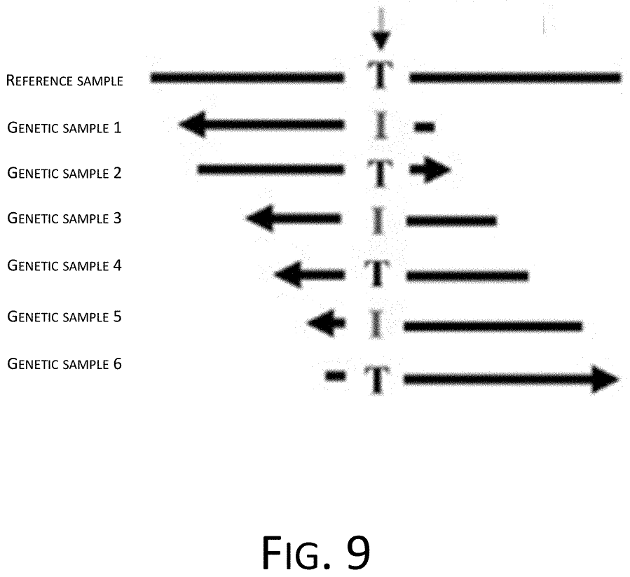

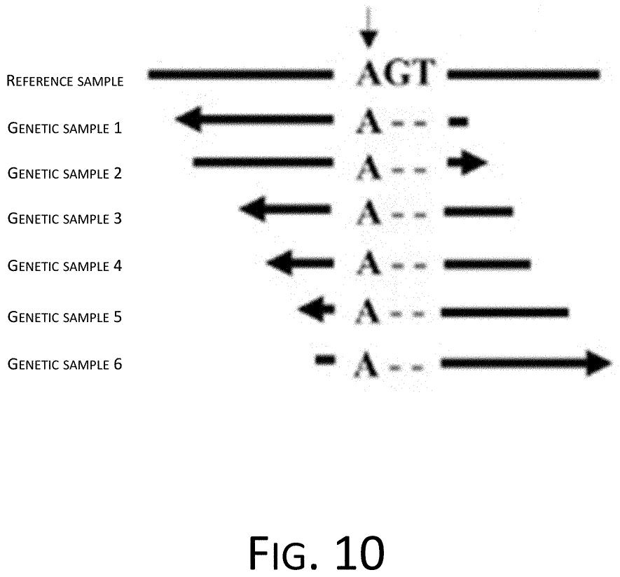

Similarly to FIG. 8, referring to FIG. 9, for a base “T” at a certain position of a reference sample, a plurality of genetic samples can be obtained by performing a plurality of forward testing and reverse testing operations, and the genetic samples are assumed to include: a genetic sample 1, a genetic sample 2, a genetic sample 3, a genetic sample 4, a genetic sample 5, and a genetic sample 6, wherein base information “I” at the corresponding positions in the genetic sample 1, the genetic sample 3 and the genetic sample 5 is different from base information “A” in the reference sample, while base information at the corresponding positions in other samples is the same as the base information in the reference sample. The proportion of the number of samples with different bases obtained by testing is relatively large, and it can thus be considered that the genetic samples obtained by testing have mutations and are the second type of genetic samples. Referring to FIG. 10, for a base “AGT” at a certain position of a reference sample, a plurality of genetic samples can be obtained by a plurality of forward testing and reverse testing operations, and the genetic samples are assumed to include: a genetic sample 1, a genetic sample 2, a genetic sample 3, a genetic sample 4, a genetic sample 5, and a genetic sample 6, wherein base information “A” at the corresponding positions in the genetic samples is different from the base information “AGT” in the reference sample, and it can thus be considered that the genetic samples obtained by testing have mutations and are the second type of genetic samples.

Continuing with the above, when the feature generation sub-model is trained and generated, genetic samples with different mutation situations may correspond to different feature generation modes. Therefore, in order to improve the quality and the effect of feature generation of the feature generation sub-model, optimization processing may be performed on the feature generation sub-model using the genetic samples with different mutation situations. Specifically, learning and training may be performed based on the genetic features corresponding to the genetic samples and the reference genetic results corresponding to the genetic samples, so as to obtain a data identification model, and the data identification model may perform variant identification operations on genetic data based on genetic features of the genetic data. It needs to be noted that the identification method of the data identification model is relatively simple, and so that a variant identification result obtained thereby is also relatively simple. Specifically, whether a mutation exists in certain genetic data can be identified, whereas a type of the mutation, a specific position of the mutation, and a degree of severity of the mutation may not need to be identified, so that the speed of operation of performing variant identification on genetic data by the data identification model is relatively high.

After the data identification model is obtained, the data identification model can be used for optimizing the feature generation sub-model, so that an optimized feature generation sub-model can be obtained. In some instances, the feature generation sub-model may include a portion of a data identification model. In this case, optimizing the feature generation sub-model using the data identification model to obtain the optimized feature generation sub-model may include: obtaining a loss function used for optimizing the data identification model; optimizing the data identification model based on the loss function to obtain an optimized data identification model; and determining the optimized feature generation sub-model based on the optimized data identification model.

Since the feature generation sub-model includes a part of the data identification model, that is, model parameters in the data identification model are the same as those in the feature generation sub-model. In this case, the feature generation sub-model can be optimized by optimizing the data identification model. In specific implementations, obtaining the loss function used for optimizing the data identification model may be performed first. In some examples, obtaining the loss function used for optimizing the data identification model may include: analyzing and processing the genetic features by using the data identification model to obtain predicted genetic results corresponding to the genetic features; determining the loss function used for optimizing the data identification model based on the genetic features, the predicted genetic results, and the reference genetic results.

Specifically, genetic samples, genetic features corresponding to the genetic samples, and reference genetic results corresponding to the genetic samples may be obtained, and then the genetic features may be analyzed using the data identification model, so that predicted genetic results corresponding to the genetic features may be obtained. A loss function used for optimizing the data identification model may then be determined based on the genetic features, the predicted genetic results, and the reference genetic results. After obtaining the loss function used for optimizing the data identification model, the data identification model may be optimized using the loss function, so that an optimized data identification model may be obtained.

In the embodiments of the present disclosure, learning and training are performed based on genetic features and reference genetic results corresponding to genetic samples to obtain a data identification model. The data identification model is then used for optimizing a feature generation sub-model to obtain an optimized feature generation sub-model, thus effectively realizing the optimization operation of the feature generation sub-model, and further improving the quality and efficiency of the feature generation sub-model on genetic data.

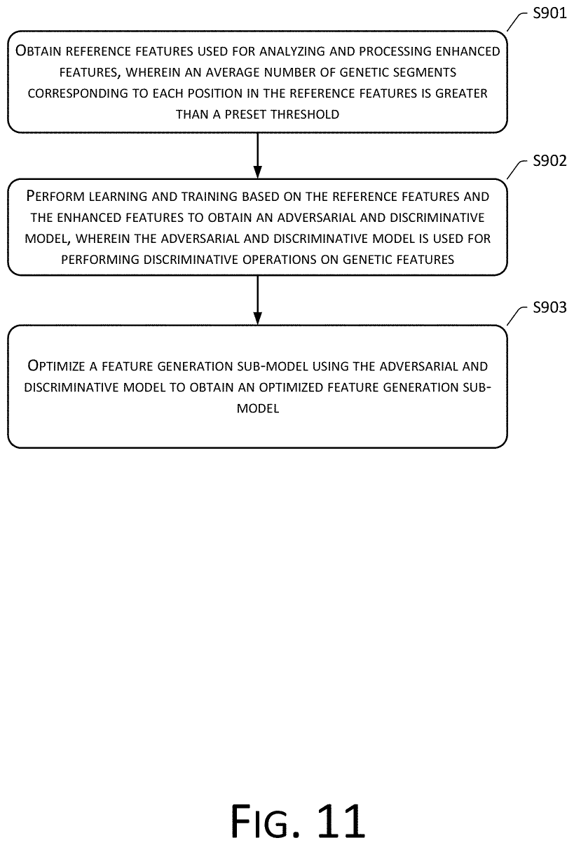

FIG. 11 is a schematic flowchart of another model training method according to the embodiments of the present disclosure. On the basis of the foregoing embodiments, referring to FIG. 11, after obtaining a feature generation sub-model, the method in the embodiments of the present disclosure may further include:

Step S901: Obtain reference features used for analyzing and processing enhanced features, wherein an average number of genetic segments corresponding to each position in the reference features is greater than a preset threshold.

Step S902: Perform learning and training based on the reference features and the enhanced features to obtain an adversarial and discriminative model, wherein the adversarial and discriminative model is used for performing discriminative operations on genetic features.

Step S903: Optimize a feature generation sub-model using the adversarial and discriminative model to obtain an optimized feature generation sub-model.

After obtaining the feature generation sub-model, the feature generation sub-model can be used to analyze genetic data, so as to obtain enhanced features corresponding to the genetic data. The enhanced features are similar to high-depth features. In order to improve the quality and efficiency of the enhanced features generated by the feature generation sub-model, the feature generation sub-model can be optimized. Specifically, reference features used for analyzing the enhanced features can be obtained, and an average number of genetic segments corresponding to each position in the reference features is larger than a preset threshold, that is, the reference features are standard high-depth features. After obtaining the reference features and the enhanced features, learning and training can be performed on the reference features and the enhanced features, that is, learning and training can be performed on association relationships between the reference feature and the enhanced features. An adversarial and discriminative model can thereby be generated, which can discriminate whether a genetic feature is a high-depth feature.

After the adversarial and discriminative model is obtained, the adversarial and discriminative model can be used for optimizing the feature generation sub-model to obtain an optimized feature generation sub-model. In some examples, optimizing the feature generation sub-model using the adversarial and discriminative model, to obtain the optimized feature generation sub-model may include: obtaining a judgment and identification result of analyzing the enhanced features by the adversarial and discriminative model; and optimizing the feature generation sub-model based on the judgment and identification result to obtain the optimized feature generation sub-model.

Specifically, after the adversarial and discriminative model is obtained, the adversarial and discriminative model may be used to analyze the enhanced features, so that a judgment and identification result of analyzing the enhanced features may be obtained. The judgment and identification result may be used to identify a degree of matching between the enhanced features and high-depth features. After the judgment and identification result is obtained, the feature generation sub-model can be optimized based on the judgment and identification result to obtain an optimized feature generation sub-model, thus further improving the quality and efficiency of analyzing and processing genetic data by the feature generation sub-model.

In the embodiments of the present disclosure, by obtaining reference features that are used for analyzing enhanced features, learning and training are then performed based on the reference features and the enhanced features to obtain an adversarial and discriminative model. A feature generation sub-model is optimized using the adversarial and discriminative model to obtain an optimized feature generation sub-model. This increases the accuracy of feature extraction operations of the feature generation sub-model on genetic data, and thereby improves the quality and efficiency of analyzing and processing the genetic data.

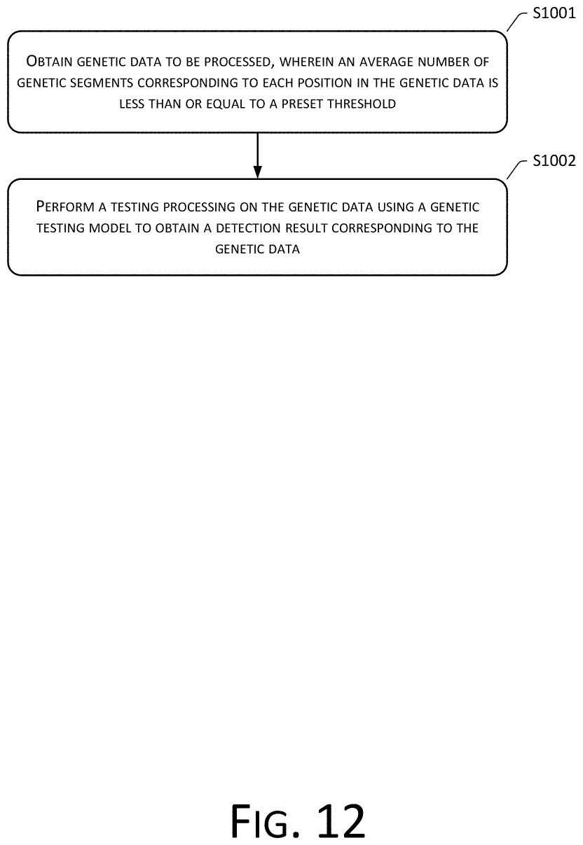

FIG. 12 is a schematic flowchart of another model training method according to the embodiments of the present disclosure. On the basis of any of the above embodiments, referring to FIG. 12, after obtaining a genetic testing model, the method in the embodiments of the present disclosure may further include:

Step S1001: Obtain genetic data to be processed, wherein an average number of genetic segments corresponding to each position in the genetic data is less than or equal to a preset threshold.

Step S1002: Perform a testing processing on the genetic data using a genetic testing model to obtain a testing result corresponding to the genetic data.

After the genetic testing model is obtained, a testing operation can be performed on the genetic data to be processed based on the genetic testing model, so that a testing result can be obtained. Specifically, the embodiments of the present disclosure do not limit specifics of implementations of performing testing processing on genetic data using a genetic testing model to obtain a testing result corresponding to the genetic data. One skilled in the art may perform settings according to specific application scenarios or application requirements. In some examples, performing testing processing on genetic data by using a genetic testing model to obtain a testing result corresponding to the genetic data may include: analyzing and processing the genetic data using the genetic testing model to obtain mutation reference information corresponding to the genetic data, wherein the mutation reference information includes at least one of the following information: 21-type genotype prediction information, zygotic prediction information, first allelic mutation length information, and second allelic mutation length information; and obtaining the testing result corresponding to the genetic data according to the mutation reference information.

After the genetic testing model and the genetic data to be processed are obtained, the genetic testing model may be used to perform testing processing on the genetic data for performing analysis processing, so that mutation reference information corresponding to the genetic data may be obtained. The mutation reference information may include at least one of: 21-type genotype prediction information, zygote prediction information, first allelic mutation length information, and second allelic mutation length information, wherein the 21-type genotypes for which the 21-type genotype prediction information is directed include: ‘AA’, ‘AC’, ‘AG’, ‘AT’, ‘CC’, ‘CG’, ‘CT’, ‘GG’, ‘GT’, ‘TT’, ‘AI’, ‘CI’, ‘GI’, ‘TI’, ‘AD’, ‘CD’, ‘GD’, ‘TD’, ‘II’, and ‘DD’, wherein A, C, G, T is four bases, and I and D are insertion and deletion respectively. The zygotic prediction information includes three types: homozygous and identical to reference base(s), homozygous and inconsistent with the reference base(s), and heterozygous. In the first allelic mutation length information, SNP mutation is 0, and Indel mutation is the length of corresponding insertion(s) and deletion(s). In the length of the second allelic mutation, SNP mutation, and Indel mutation is the length of corresponding insertion(s) and deletion(s).

After obtaining the mutation reference information corresponding to the genetic data, the mutation reference information may be analyzed to obtain a testing result. It is understood that the testing result is obtained based on at least one of the 21-type genotype prediction information, the zygote prediction information, the first allelic mutation length information, and the second allelic mutation length information, thereby ensuring the accuracy and reliability of determining the testing result.

In still other examples, after obtaining the testing result corresponding to the genetic data, the method of the embodiments of the present disclosure may further include: performing disease prediction based on the testing result.

When a mutation condition exists in the genetic data, this indicates that an object (a human or animal) is prone to getting a related disease. In this case, disease prediction may be performed based on the testing result. Specifically, probability information of a set object getting a related disease may be determined based on the mutation condition of the genetic data. It is understood that the probability information is related to the extent of mutation of an associated gene sequence, and the higher the extent of mutation is, the higher the probability is. The lower the extent of mutation is, the lower the probability is. Conversely, an absence of a mutation condition in a gene sequence indicates that a set object is not likely to get a related disease.

According to the technical solutions provided by the embodiments of the present disclosure, by obtaining genetic data to be processed and performing testing processing on the genetic data using a genetic testing model to obtain a testing result corresponding to the genetic data, not only the accuracy of the genetic testing operation is ensured, but also the cost and amount of data processing are effectively reduced, thus effectively realizing more accurate testing operations based on low-depth genetic data, which further improves the practicability of the method, and facilitates the popularization and applications thereof in the market.

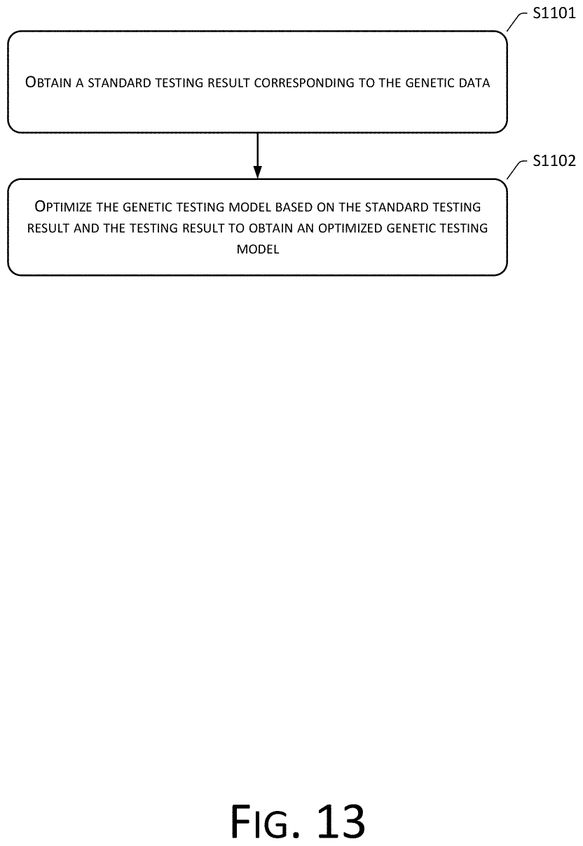

FIG. 13 is a schematic flowchart of another model training method according to the embodiments of the present disclosure. On the basis of the above embodiments, referring to FIG. 13, after obtaining a testing result corresponding to genetic data, the method in the embodiments of the present disclosure may further include:

Step S1101: Obtain a standard testing result corresponding to the genetic data.

Step S1102: Optimize the genetic testing model based on the standard testing result and the testing result to obtain an optimized genetic testing model.

After genetic data is obtained, the genetic data can be analyzed and processed using a genetic testing model, so that a testing result can be obtained. In order to improve the quality and efficiency of analysis processing of genetic data by the genetic testing model, the genetic testing model may be optimized in a regular or irregular basis. Specifically, a standard testing result corresponding to the genetic data can be obtained, then the genetic testing model can be optimized based on the standard testing result and the testing result. Specifically, a degree of matching between the standard testing result and the testing result can be identified, and then the genetic testing model is optimized based on the degree of matching, so that an optimized genetic testing model can be obtained. In this way, when the optimized genetic testing model is used for analyzing and processing genetic data, the quality and efficiency of data processing can be effectively improved.

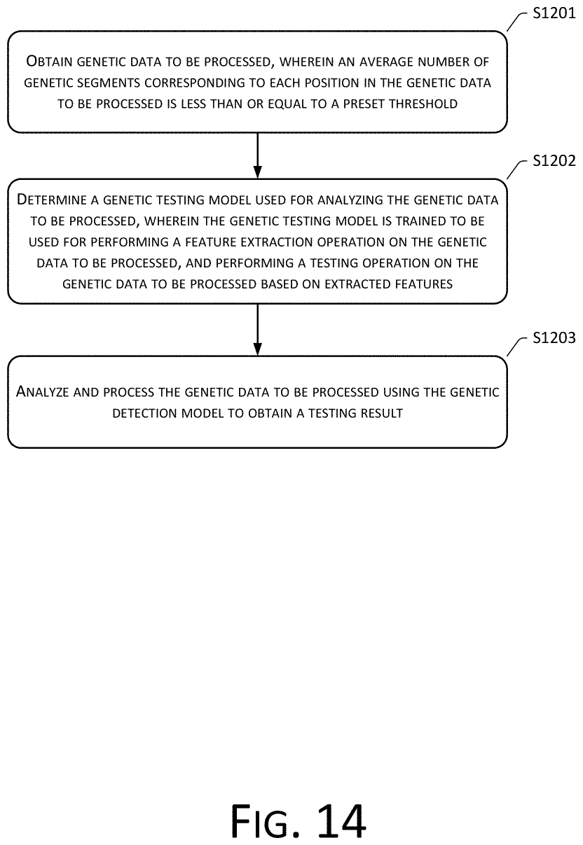

FIG. 14 is a schematic flowchart of a genetic testing method according to the embodiments of the present disclosure. Referring to FIG. 14, the embodiments of the present disclosure provide a genetic testing method. An execution subject of the method can be a genetic testing apparatus. It can be understood that the genetic testing apparatus can be implemented as software, or a combination of software and hardware. Specifically, the genetic testing method can include the following steps:

Step S1201: Obtain genetic data to be processed, wherein an average number of genetic segments corresponding to each position in the genetic data to be processed is less than or equal to a preset threshold.

Specific implementations and implementation effects of “obtaining genetic data to be processed” in the embodiments of the present disclosure are similar to those of step S101 in the above embodiments, and details thereof may be referenced to the above description, and are not repeated herein.

Step S1202: Determine a genetic testing model used for analyzing the genetic data to be processed, wherein the genetic testing model is trained to be used for performing a feature extraction operation on the genetic data to be processed, and performing a testing operation on the genetic data to be processed based on extracted features.

The genetic testing model is obtained by performing learning and training based on a full convolutional neural network, and the full convolutional neural network can be a two-dimensional network model or a three-dimensional network model. The genetic testing model can perform a feature extraction operation on the genetic data to be processed and perform a testing operation on the genetic data to be processed based on extracted features, so that a relatively accurate genetic testing operation on the genetic data is effectively realized.

Step S1203: Analyze and process the genetic data to be processed using the genetic testing model to obtain a testing result.

After the genetic testing model and the genetic data to be processed are obtained, the genetic testing model can be used for analyzing and processing the genetic data to be processed, so that a testing result can be obtained. In some examples, to improve the practicability of the method, after obtaining the testing result, the method in the embodiments of the present disclosure may further include: performing disease prediction based on the testing result.

When a mutation condition exists in the genetic data, this indicates that a set object is prone to getting a related disease. In this case, disease prediction may be performed based on the testing result. Specifically, probability information of a set object getting a related disease may be determined based on the mutation condition of the genetic data. Tt is understood that the probability information is related to the extent of mutation of an associated gene sequence, and the higher the extent of mutation is, the higher the probability is. The lower the extent of mutation is, the lower the probability is. Conversely, an absence of a mutation condition in a gene sequence indicates that a set object is not likely to get a related disease.

By obtaining genetic data to be processed, the genetic testing method provided by the embodiments of the present disclosure determines a genetic testing model used for analyzing and processing the genetic data to be processed, and then analyzes and processes the genetic data to be processed using the genetic testing model, thereby realizing a feature extraction operation through the low-depth genetic data to be processed to obtain genetic features. The genetic testing method then enhances the genetic features to obtain enhanced features corresponding to the genetic features, and perform testing on the genetic data based on the enhanced features to obtain a testing result. This thus not only ensures the accuracy of genetic testing operations, but also effectively reduces the cost and amount of data processing. Therefore, relatively accurate testing operations can be effectively realized based on low-depth genetic data, the practicability of the method is further improved, and the method is favorable for popularization and applications in the market.

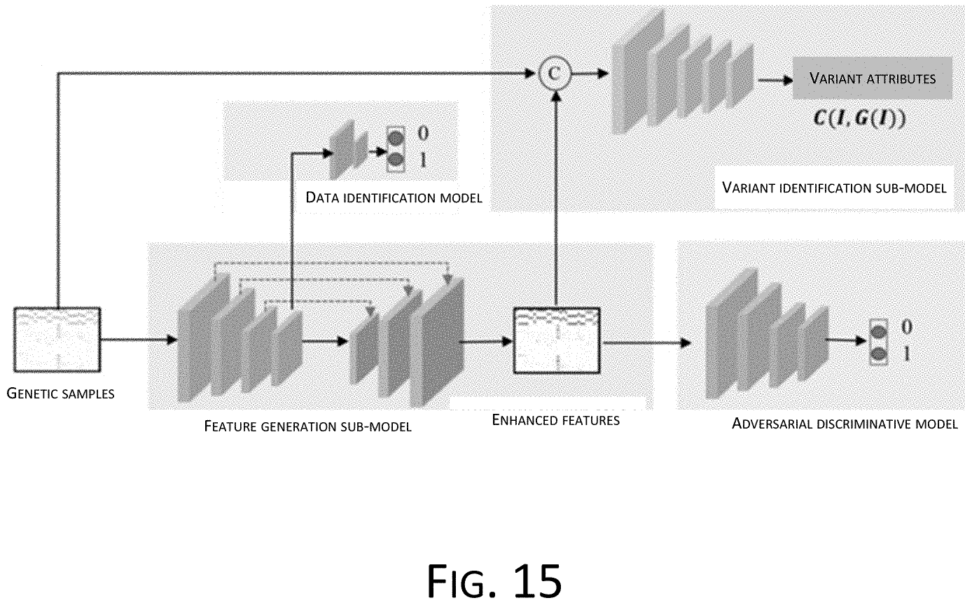

In specific applications, referring to FIG. 15, the embodiments of the present application provides a genetic testing method. The genetic testing method may include: a model training process and a testing process. Specifically, a genetic testing model generated from training may include: a feature generation sub-model and a variant identification sub-model. The model training process includes the following steps:

Step 101: Obtain genetic samples, wherein the genetic samples have corresponding sample mutation results, and an average number of genetic segments corresponding to each position in the genetic samples is less than or equal to a preset threshold.

Step 102: Determine genetic features corresponding to the genetic samples and enhanced features corresponding to the genetic features.

Step 103: Perform learning and training based on the genetic samples, the genetic features and the enhanced features to obtain a feature generation sub-model, wherein the feature generation sub-model is used for performing feature extraction and performing enhancement processing on extracted genetic features.

The generated feature generation sub-model can extract low-depth sequencing data from the genetic samples, and generate a feature map of high-depth sequencing data from the low-depth sequencing data, which can specifically be realized as a 2-dimensional full convolutional network model.

Step 104: Perform learning and training on the genetic features and the reference genetic results corresponding to the genetic samples after the feature generation sub-model is obtained to obtain a data identification model, the data identification model being used for performing a variant identification operation on the genetic data based on the genetic features.

Step 105: Use the data identification model for analyzing and processing the genetic features to obtain predicted genetic results corresponding to the genetic features, and determining a loss function used for optimizing the data identification model based on the genetic features, the predicted genetic results, and the reference genetic results wherein the feature generation sub-model includes a part of the data identification model.

Specifically, the data identification model can identify whether the genetic data are variant data based on the genetic features. Since the feature generation sub-model is used for generating enhanced features which are close to individual pixels in high-depth features, the feature generation sub-model has no identifying capability on whether data are variant data, where the identifying capability is conductive to improving the accuracy of feature point generation tasks and reducing the false sample rate of the model. In addition, the main network of the data identification model is the same as a partial network corresponding to an encoder in the feature generation sub-model. Therefore, the feature generation sub-model can be optimized by optimizing the data identification model, and the quality and the effect of feature generation can be promoted.

Step 106: Optimize the data identification model based on the loss function to obtain an optimized data identification model, and determine an optimized feature generation sub-model based on the optimized data identification model.

Step 107: Perform learning and training based on the enhanced features and the reference genetic results corresponding to the genetic samples to obtain a variant identification model, wherein the variant identification model is used for performing variant testing operations on genetic data based on feature information.

Step 108: Obtain reference features used for analyzing and processing the enhanced features, wherein the reference features are high-depth data features, and perform learning and training on the reference features and the enhanced features to obtain an adversarial discriminative model.

Step 109: Optimize the feature generation sub-model using the adversarial discriminative model to obtain an optimized feature generation sub-model.

The adversarial discriminative model is used for identifying a degree of matching between a real high-depth feature map and a predicted high-depth feature map. The feature generation sub-model and the genetic testing model have an adversarial relationship. Introducing the adversarial discriminative model can promote the accuracy of data analysis of the feature generation sub-model.

Step 110: Generate a genetic testing model based on the feature generation sub-model and the variant identification sub-model.

After the genetic testing model is generated by training, the genetic testing model can be used for analyzing and processing genetic data so as to realize variant testing operations. Specifically, the method includes the following steps:

Step 201: Obtain genetic data to be processed, wherein an average number of genetic segments corresponding to each position in the genetic data is less than or equal to a preset threshold.

Step 202: Perform mutation testing processing on the genetic data using a genetic testing model to obtain a mutation testing result corresponding to the genetic data.

Step 203: Obtain a standard testing result corresponding to the genetic data, and optimize the genetic testing model based on the standard testing result and the mutation testing result to obtain an optimized genetic testing model.

According to the technical solutions, a framework corresponding to the genetic testing model that is generated by training is generated by end-to-end training. This can enable the optimization and training of the genetic testing model to have a better effect. Specifically, the feature generation sub-model has the capability of identifying whether a variant exists in data by introducing the data identification model, and the quality and the effect of genetic feature generation are further improved. In addition, the variant identification sub-model is optimized by introducing the adversarial discriminative model and adopting a mutual promotion mode. This facilitates the effect of generating a genetic mutation testing result, thus further improving the practicability of the method, and facilitating the popularization and the application thereof in the market.

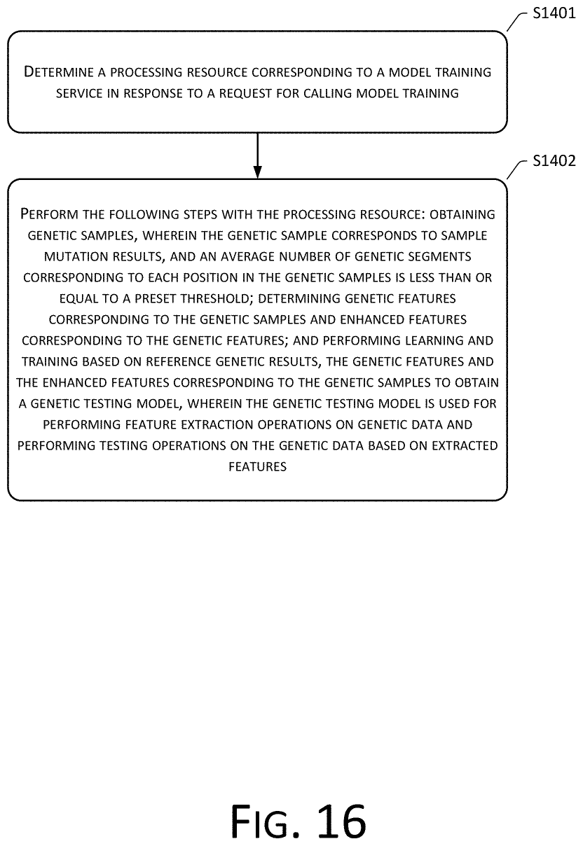

FIG. 16 is a schematic flowchart of another model training method according to the embodiments of the present disclosure. Referring to FIG. 16, the embodiments provide another model training method. An execution subject of the model training method may be a model training apparatus. The model training apparatus may be implemented as software, or a combination of software and hardware. Specifically, the model training method may include the following steps:

Step S1401: Determine a processing resource corresponding to a model training service in response to a request for calling model training.

Step S1402: Perform the following steps with the processing resource: obtaining genetic samples, wherein the genetic sample corresponds to sample mutation results, and an average number of genetic segments corresponding to each position in the genetic samples is less than or equal to a preset threshold; determining genetic features corresponding to the genetic samples and enhanced features corresponding to the genetic features; and performing learning and training based on reference genetic results, the genetic features and the enhanced features corresponding to the genetic samples to obtain a genetic testing model, wherein the genetic testing model is used for performing feature extraction operations on genetic data and performing testing operations on the genetic data based on extracted features.

Specifically, the model training method provided by the present disclosure can be executed at the cloud end. A plurality of computing nodes can be deployed at the cloud end, and each computing node has processing resources such as computation, storage and the like. In the cloud, multiple computing nodes may be organized to provide a service, and apparently, a single computing node may also provide one or more services.

For the solutions provided by the present disclosure, the cloud end can provide a service for completing the model training method, which is called as a model training service. When a user needs to use the model training service, the user invokes the model training service to trigger a request for calling the model training service to the cloud. The request may include genetic samples. The cloud determines a computing node that responds to the request, and performs the following steps using processing resources in the computing node: obtaining genetic samples, wherein the genetic sample corresponds to sample mutation results, and an average number of genetic segments corresponding to each position in the genetic samples is less than or equal to a preset threshold; determining genetic features corresponding to the genetic samples and enhanced features corresponding to the genetic features; and performing learning and training based on reference genetic results, the genetic features and the enhanced features corresponding to the genetic samples to obtain a genetic testing model, wherein the genetic testing model is used for performing feature extraction operations on genetic data and performing testing operations on the genetic data based on extracted features.

Specifically, the processes, principles and effects of implementations of the above method steps in the embodiments of the present disclosure are similar to the processes, principles and effects of implementations of the above method steps in the embodiments as shown in FIGS. 4-13 and FIG. 15. For parts not described in detail in the embodiments of the present disclosure, reference may be made to related descriptions of the embodiments as shown in FIGS. 4-13 and FIG. 15.

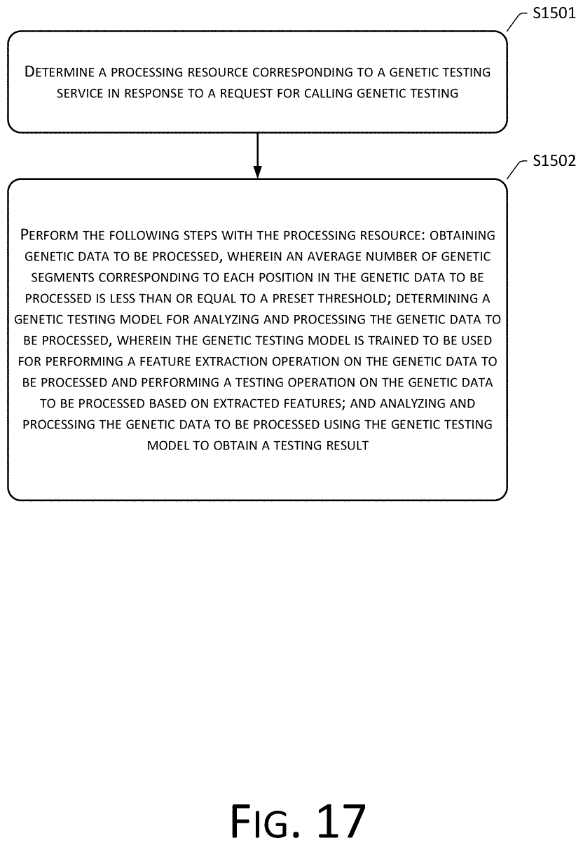

FIG. 17 is a schematic flowchart showing another genetic testing method according to the embodiments of the present disclosure. Referring to FIG. 17, the embodiments of the present disclosure provide another genetic testing method. An execution subject of the genetic testing method may be a genetic testing apparatus. The genetic testing apparatus may be implemented as software, or a combination of software and hardware. Specifically, the model training method may include the following steps:

Step S1501: Determine a processing resource corresponding to a genetic testing service in response to a request for calling genetic testing.

Step S1502: Perform the following steps with the processing resource: obtaining genetic data to be processed, wherein an average number of genetic segments corresponding to each position in the genetic data to be processed is less than or equal to a preset threshold; determining a genetic testing model for analyzing and processing the genetic data to be processed, wherein the genetic testing model is trained to be used for performing a feature extraction operation on the genetic data to be processed and performing a testing operation on the genetic data to be processed based on extracted features; and analyzing and processing the genetic data to be processed using the genetic testing model to obtain a testing result.

Specifically, the genetic testing method provided by the present disclosure can be executed at the cloud end. A plurality of computing nodes can be deployed at the cloud end, and each computing node has processing resources such as computation, storage and the like. In the cloud, multiple computing nodes may be organized to provide a service, and apparently, a single computing node may also provide one or more services.

For the solutions provided by the present disclosure, the cloud end can provide a service for completing the genetic testing method, which is called as a genetic testing service. When a user needs to use the genetic testing service, the user invokes the genetic testing service to trigger a request for calling the genetic testing service to the cloud. The request may include genetic data to be processed. The cloud determines a computing node that responds to the request, and performs the following steps using processing resources in the computing node: obtaining genetic data to be processed, wherein an average number of genetic segments corresponding to each position in the genetic data to be processed is less than or equal to a preset threshold; determining a genetic testing model for analyzing and processing the genetic data to be processed, wherein the genetic testing model is trained to be used for performing a feature extraction operation on the genetic data to be processed and performing a testing operation on the genetic data to be processed based on extracted features; and analyzing and processing the genetic data to be processed using the genetic testing model to obtain a testing result.

Specifically, the processes, principles and effects of implementations of the above method steps in the embodiments of the present disclosure are similar to the processes, principles and effects of implementations of the above method steps in the embodiments as shown in FIGS. 14 and 15. For parts not described in detail in the embodiments of the present disclosure, reference may be made to related descriptions of the embodiments as shown in FIGS. 14 and 15.

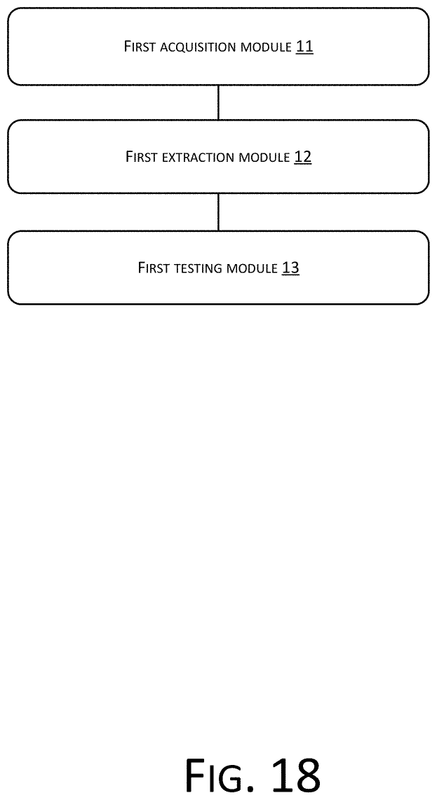

FIG. 18 is a schematic structural diagram of a genetic testing device according to the embodiments of the present disclosure. Referring to FIG. 18, the embodiments of the present disclosure provide a genetic testing apparatus that can be used to perform the genetic testing method as shown in FIG. 3. Specifically, the genetic testing apparatus can include: a first acquisition module 11, a first extraction module 12 and a first testing module 13, wherein:

the first acquisition module 11 is configured to obtain genetic data to be processed, wherein an average number of genetic segments corresponding to each position in the genetic data to be processed is less than or equal to a preset threshold;

the first extraction module 12 is configured to input the genetic data to be processed to a feature generation network layer for performing a feature extraction operation to obtain genetic features corresponding to the genetic data to be processed and enhanced features corresponding to the genetic features; and

the first testing module 13 is configured to input the genetic data to be processed and the enhanced features into a genetic identification network layer for performing a genetic testing operation to obtain a testing result.

In some examples, when the first testing module 13 inputs the genetic data to be processed and the enhanced features into the genetic identification network layer for performing the genetic testing operation to obtain the testing result, the first testing module 13 is configured to perform: performing genetic testing processing on the genetic data to be processed and the enhanced features using the genetic identification network layer to obtain testing reference information corresponding to the genetic data to be processed, wherein the testing reference information includes at least one of the following information: 21-type genotype prediction information, zygotic prediction information, first allelic mutation length information, and second allelic mutation length information; and obtaining the testing result corresponding to the genetic data to be processed according to the testing reference information.

In some examples, the first acquisition module 11 and the first testing module 13 in the embodiments of the present disclosure are configured to perform the following steps:

the first acquisition module 11 configured to obtain a standard data type corresponding to the genetic data to be processed; and

the first testing module 13 configured to input the genetic features to the data identification network layer for performing a data type identification operation to obtain a genetic data type; determining a loss function used for the feature generation network layer based on the genetic data type and the standard data type; and optimizing the feature generation network layer using the loss function to obtain an optimized feature generation network layer.

In some examples, the feature generation network layer includes a portion of the data identification network layer, and when the first testing module 13 optimizes the feature generation network layer using the loss function to obtain the optimized feature generation network layer, the first testing module 13 is configured to perform: optimizing the data identification network layer based on the loss function to obtain an optimized data identification network layer; and determining an optimized feature generation network layer based on the optimized data identification network layer.

The apparatus shown in FIG. 18 can perform the methods of the embodiments shown in FIG. 1 and FIG. 3. For parts not described in detail in the embodiments of the present disclosure, reference may be made to related descriptions of the embodiments shown in FIG. 1 and FIG. 3. The implementation processes and technical effects of this technical solution can be referenced to the descriptions in the embodiments shown in FIG. 1 and FIG. 3, and are not described herein again.

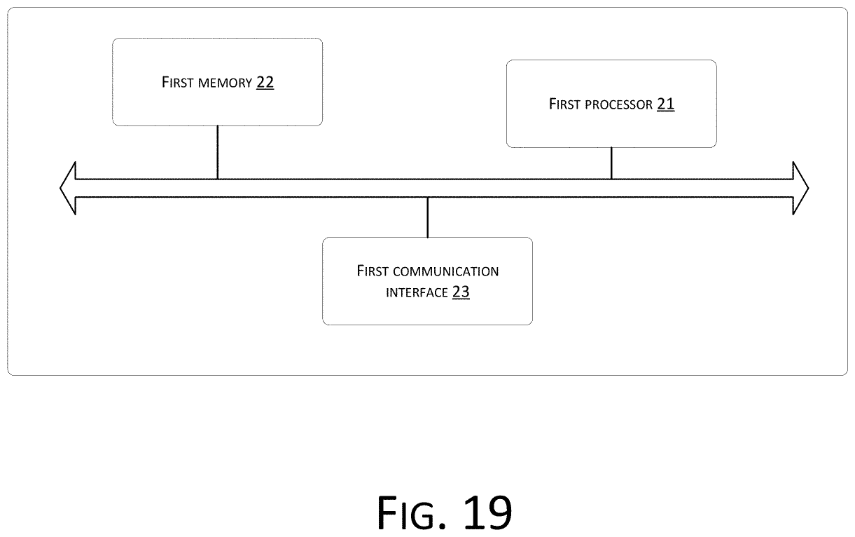

In a possible design, the structure of the genetic testing apparatus shown in FIG. 18 may be implemented as an electronic device. The electronic device may be a variety of types of devices, such as a mobile phone, a tablet computer, a server, etc. As shown in FIG. 19, the electronic device may include: a first processor 21 and a first memory 22. The first memory 22 is configured to store programs for executing the genetic testing methods provided in the embodiments shown in FIGS. 1 and 3 by a corresponding electronic device, and the first processor 21 is configured to execute the programs stored in the first memory 22.

A program includes one or more computer instructions, wherein the one or more computer instructions, when executed by the first processor 21, are capable of performing the following steps:

obtaining genetic data to be processed, wherein an average number of genetic segments corresponding to each position in the genetic data to be processed is less than or equal to a preset threshold;

inputting the genetic data to be processed into a feature generation network layer for performing a feature extraction operation to obtain genetic features corresponding to the genetic data to be processed and enhanced features corresponding to the genetic features; and

inputting the genetic data to be processed and the enhanced features into a genetic identification network layer for performing a genetic testing operation to obtain a testing result.

Further, the first processor 21 is also configured to perform all or part of the steps in the embodiments shown in FIG. 1 and FIG. 3.

The electronic device may further include a first communication interface 23 which is used by the electronic device for communicating with other devices or a communication network.

In addition, the embodiments of the present disclosure provide a computer storage medium configured to store computer software instructions that are used by an electronic device, which include programs for executing the genetic testing methods in the method embodiments shown in FIGS. 1 and 3.

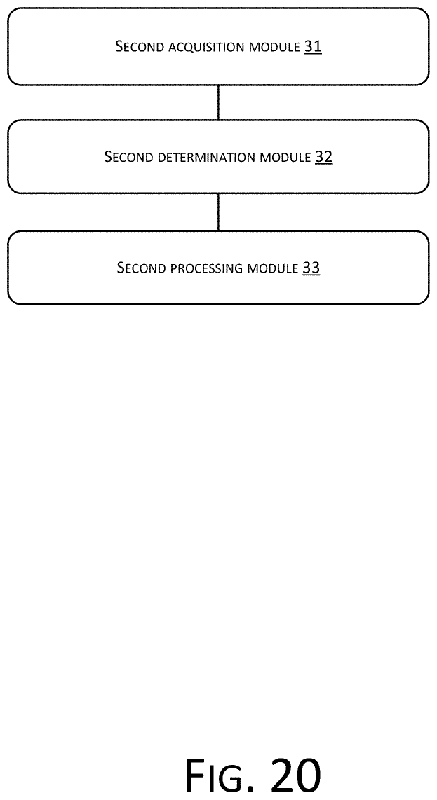

FIG. 20 is a schematic structural diagram of a model training apparatus according to the embodiments of the present disclosure. Referring to FIG. 20, the embodiments of the present disclosure provide a model training apparatus, which can perform the model training method shown in FIG. 4. Specifically, the model training apparatus can include: a second acquisition module 31, a second determination module 32, and a second processing module 33, wherein:

the second acquisition module 31 is configured to obtain genetic samples, where the genetic samples correspond to sample mutation results, and an average number of genetic segments corresponding to each position in the genetic samples is less than or equal to a preset threshold;

the second determination module 32 is configured to determine genetic features corresponding to the genetic samples and enhanced feature corresponding to the genetic features; and

the second processing module 33 is configured to perform learning and training based on reference genetic results, the genetic features, and the enhanced features corresponding to the genetic samples to obtain a genetic testing model, wherein the genetic testing model is configured to perform a feature extraction operation on genetic data and perform a testing operation on the genetic data based on extracted features.

In some examples, when the second processing module 33 performs learning and training based on the reference genetic results, the genetic features and the enhanced features corresponding to the genetic samples to obtain the genetic testing model, the second processing module 33 is configured to perform: performing learning and training based on the genetic samples, the genetic features and the enhanced features to obtain a feature generation sub-model, wherein the feature generation sub-model is used for performing feature extraction and enhancing extracted genetic features; performing learning and training based on the enhanced features and the reference genetic results corresponding to the genetic samples to obtain a variant identification model, wherein the variant identification model is used for testing genetic data based on feature information; and generating the genetic testing model based on the feature generation sub-model and the variant identification sub-model.

In some examples, after obtaining the feature generation sub-model, the second processing module 33 in the embodiments of the present disclosure may be further configured to: performing learning and training based on the genetic features and the reference genetic results corresponding to the genetic samples to obtain a data identification model, wherein the data identification model is used for performing a variant identification operation on genetic data based on genetic features; and optimizing the feature generation sub-model using the data identification model to obtain an optimized feature generation sub-model.

In some examples, the feature generation sub-model includes a portion of the data identification model, and when the second processing module 33 optimizes the feature generation sub-model using the data identification model to obtain the optimized feature generation sub-model, the second processing module 33 is configured to perform: obtaining a loss function used for optimizing the data identification model; optimizing the data identification model based on the loss function to obtain an optimized data identification model; and determining the optimized feature generation sub-model based on the optimized data identification model.

In some examples, when the second processing module 33 obtains the loss function for optimizing the data identification model, the second processing module 33 is configured to perform: analyzing and processing the genetic features using a data identification model to obtain predicted genetic results corresponding to the genetic features; and determining a loss function used for optimizing the data identification model based on the genetic features, the predicted genetic results, and the reference genetic results.

In some examples, after obtaining the feature generation sub-model, the second acquisition module 31 and the second processing module 33 in the embodiments of the present disclosure are configured to perform the following steps:

the second acquisition module 31 configured to obtain reference features for performing analysis processing on the enhanced features, wherein an average number of genetic segments corresponding to each position in the reference features is greater than the preset threshold; and

the second processing module 33 configured to perform learning and training based on the reference features and the enhanced features to obtain an adversarial discriminative model, wherein the adversarial discriminative model is configured to perform a discriminative operation on the genetic features; and optimizing the feature generation sub-model using the adversarial discriminative model to obtain an optimized feature generation sub-model.

In some examples, when the second processing module 33 optimizes the feature generation sub-model using the adversarial discriminative model to obtain the optimized feature generation sub-model, the second processing module 33 is configured to perform: obtaining a judgment and identification result of analyzing and processing the enhanced features using the adversarial discriminative model; and optimizing the feature generation sub-model based on the judgment and identification result to obtain the optimized feature generation sub-model.

In some examples, after obtaining the genetic testing model, the second acquisition module 31 and the second processing module 33 in the embodiments of the present disclosure are configured to perform the following steps:

the second acquisition module 31 configured to obtain genetic data to be processed, where an average number of genetic segments corresponding to each position in the genetic data is less than or equal to the preset threshold; and

the second processing module 33 configured to perform testing processing on the genetic data using the genetic testing model to obtain a testing result corresponding to the genetic data.

In some examples, when the second processing module 33 performs the testing processing on the genetic data using the genetic testing model to obtain the testing result corresponding to the genetic data, the second processing module 33 is configured to perform: analyzing and processing the genetic data using the genetic testing model to obtain mutation reference information corresponding to the genetic data, wherein the mutation reference information includes at least one of the following information: 21-type genotype prediction information, zygotic prediction information, first allelic mutation length information, and second allelic mutation length information; and obtaining the testing result corresponding to the genetic data according to the mutation reference information.

In some examples, after obtaining the testing result corresponding to the genetic data, the second acquisition module 31 and the second processing module 33 in the embodiments of the present disclosure are configured to perform the following steps:

the second acquisition module 31 configured to obtain a standard testing result corresponding to the genetic data; and

the second processing module 33 configured to optimize the genetic testing model based on the standard testing result and the testing result, and obtain an optimized genetic testing model.

In some examples, after obtaining the testing result corresponding to the genetic data, the second processing module 33 in the embodiments of the present disclosure is configured to perform the following steps: performing disease prediction based on the testing result.

The apparatus shown in FIG. 20 can perform the methods of the embodiments shown in FIG. 2, FIGS. 4-13, and FIG. 15. For parts that are not described in detail in the embodiments of the present disclosure may be referenced to related descriptions of the embodiments shown in FIG. 2, FIGS. 4-13, and FIG. 15. The implementation processes and technical effects of the technical solution are described in the embodiments shown in FIG. 2, FIGS. 4-13, and FIG. 15, and are not repeatedly described herein.

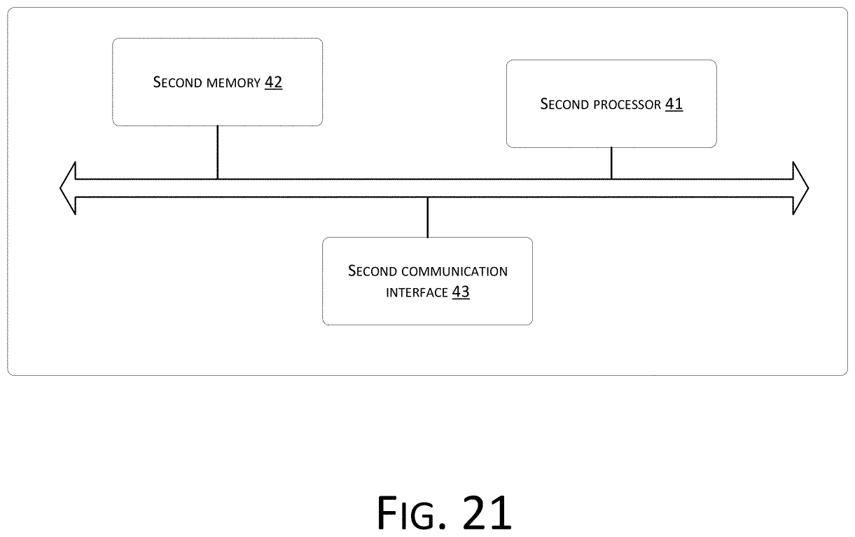

In a possible design, the structure of the model training apparatus shown in FIG. 20 may be implemented as an electronic device. The electronic device may be a variety of types of devices such as a mobile phone, a tablet computer, a server, etc. As shown in FIG. 21, the electronic device may include: a second processor 41 and a second memory 42. The second memory 42 is configured to store programs for executing the model training methods provided in the embodiments shown in FIG. 2, FIGS. 4-13, and FIG. 15 by a corresponding electronic device, and the second processor 41 is configured to execute the programs stored in the second memory 42.

A program includes one or more computer instructions, wherein the one or more computer instructions, when executed by the second processor 41, are capable of performing the following steps:

obtaining genetic samples, wherein the genetic samples correspond to sample mutation results, and an average number of genetic segments corresponding to each position in the genetic samples is less than or equal to a preset threshold;

determining genetic features corresponding to the genetic samples and enhanced features corresponding to the genetic features; and

performing learning and training based on reference genetic results, the genetic features and the enhanced features corresponding to the genetic samples to obtain a genetic testing model, wherein the genetic testing model is used for performing feature extraction operations on genetic data and performing testing operations on the genetic data based on extracted features.

Further, the second processor 41 is also used to perform all or part of the steps in the embodiments shown in FIG. 2, FIGS. 4-13, and FIG. 15.

The electronic device may further include a second communication interface 43 which is used by the electronic device for communicating with other devices or a communication network.

In addition, the embodiments of the present disclosure provide a computer storage medium configured to store computer software instructions used by an electronic device, which include programs for executing the model training methods in the method embodiments shown in FIG. 2, FIGS. 4-13, and FIG. 15.

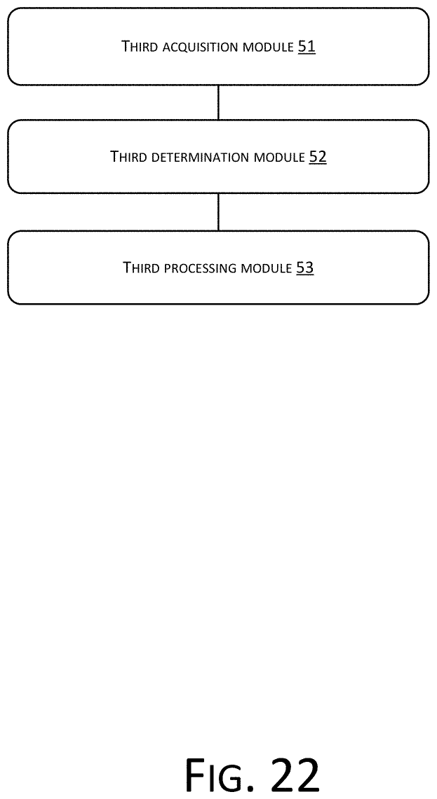

FIG. 22 is a schematic structural diagram of a genetic testing apparatus according to the embodiments of the present disclosure. Referring to FIG. 22, the embodiments of the present disclosure provide a genetic testing apparatus that can perform the genetic testing method shown in FIG. 14 described above. The genetic testing apparatus can include: a third acquisition module 51, a third determination module 52 and a third processing module 53, wherein:

the third acquisition module 51 configured to obtain genetic data to be processed, wherein an average number of genetic segments corresponding to each position in the genetic data to be processed is less than or equal to a preset threshold;

the third determination module 52 configured to determine a genetic testing model used for analyzing and processing the genetic data to be processed, where the genetic testing model is trained to perform a feature extraction operation on the genetic data to be processed, and perform a testing operation on the genetic data to be processed based on extracted features; and

the third processing module 53 configured to analyze and process the genetic data to be processed using the genetic testing model to obtain a testing result.

The apparatus shown in FIG. 22 can perform the methods of the embodiments shown in FIGS. 14 and 15. For parts that are not described in detail in the embodiments of the present disclosure may be referenced to related descriptions of the embodiments shown in FIGS. 14 and 15. The implementation processes and technical effects of the technical solution are described in the embodiments shown in FIGS. 14 and 15, and are not repeatedly described herein.

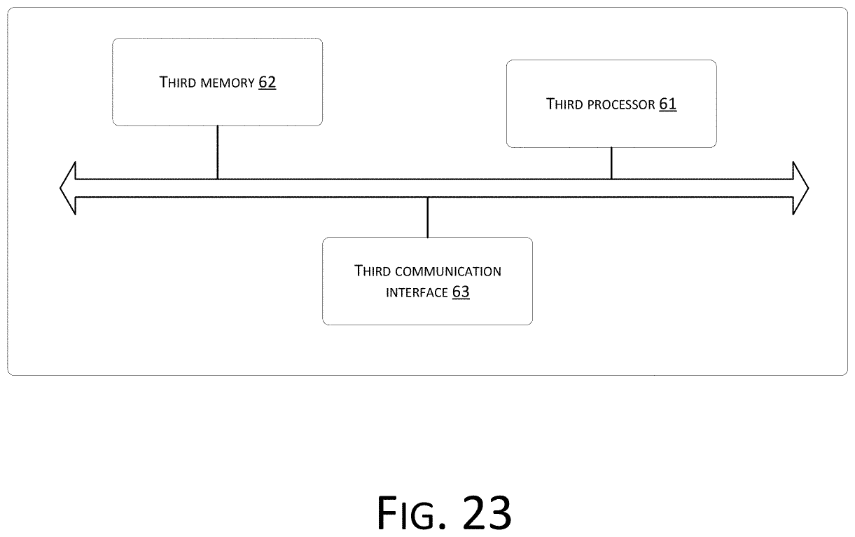

In a possible design, the structure of the genetic testing apparatus shown in FIG. 22 can be implemented as an electronic device. The electronic device may be a variety of types of devices, such as a mobile phone, a tablet computer, a server, etc. As shown in FIG. 23, the electronic device may include: a third processor 61 and a third memory 62. The third memory 62 is configured to store a program for executing the genetic testing method provided in the embodiments shown in FIG. 14 by a corresponding electronic device, and the third processor 61 is configured to execute the program stored in the third memory 62.

A program includes one or more computer instructions, wherein the one or more computer instructions, when executed by the third processor 61, are capable of performing the following steps:

obtaining genetic data to be processed, wherein an average number of genetic segments corresponding to each position in the genetic data to be processed is less than or equal to a preset threshold;

determining a genetic testing model for analyzing and processing the genetic data to be processed, wherein the genetic testing model is trained to be used for performing a feature extraction operation on the genetic data to be processed and performing a testing operation on the genetic data to be processed based on extracted features; and

analyzing and processing the genetic data to be processed using the genetic testing model to obtain a testing result.

Further, the third processor 61 is also configured to perform all or part of the steps in the embodiments shown in FIG. 14.

The electronic device may further include a third communication interface 63 which is used by the electronic device for communicating with other devices or a communication network.

In addition, the embodiments of the present disclosure provide a computer storage medium configured to store computer software instructions used by an electronic device, which include programs for executing the genetic testing method in the embodiments of the method shown in FIG. 14.

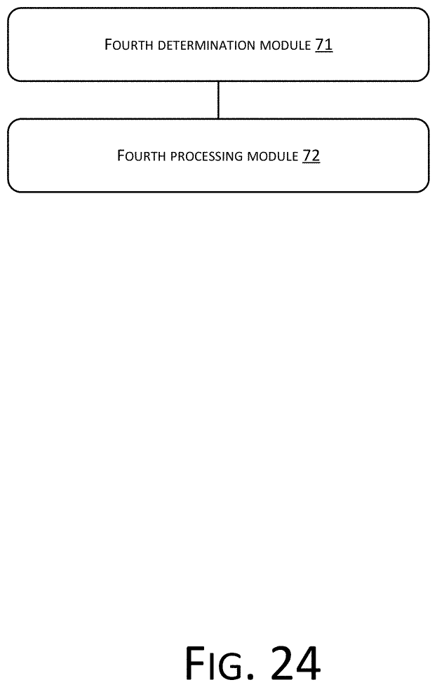

FIG. 24 is a schematic structural diagram of another model training apparatus according to the embodiments of the present disclosure. Referring to FIG. 24, the embodiments of the present disclosure provide another model training apparatus that can perform the model training method shown in FIG. 16. The model training apparatus may include: a fourth determination module 71 and a fourth processing module 72, wherein:

the fourth determination module 71 configured to determine a processing resource corresponding to a model training service in response to a request for calling model training;

the fourth processing module 72 configured to perform the following steps with the processing resource: obtaining genetic samples, wherein the genetic samples correspond to sample mutation results, and an average number of genetic segments corresponding to each position in the genetic samples is less than or equal to a preset threshold; determining genetic features corresponding to the genetic samples and enhanced features corresponding to the genetic features; and performing learning and training based on reference genetic results, the genetic features and the enhanced features corresponding to the genetic samples to obtain a genetic testing model, wherein the genetic testing model is used for performing feature extraction operations on genetic data and performing testing operations on the genetic data based on extracted features.

The apparatus shown in FIG. 24 can execute the method of the embodiments shown in FIG. 16. For parts that are not described in detail in the embodiments of the present disclosure may be referenced to related descriptions of the embodiments shown in FIG. 14. The implementation processes and technical effects of the technical solution are described in the embodiments shown in FIG. 14, and are not repeatedly described herein.

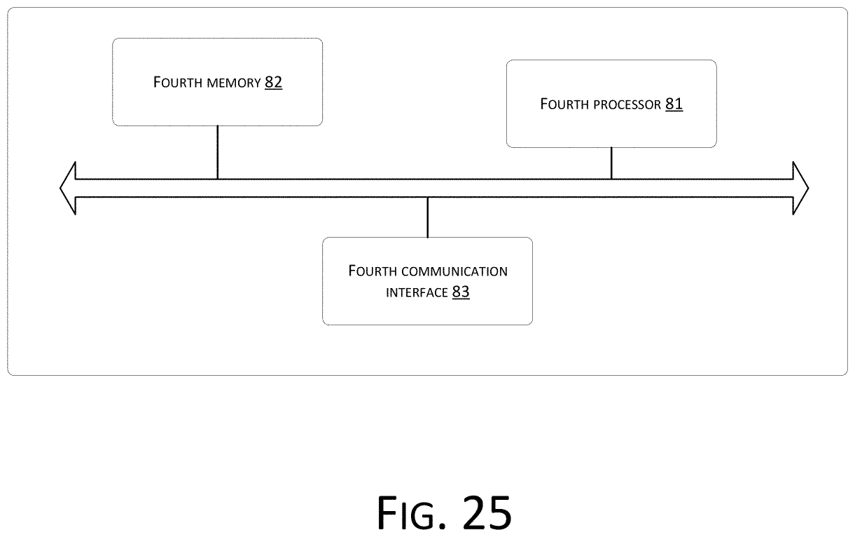

In a possible design, the structure of the model training apparatus shown in FIG. 24 may be implemented as an electronic device. The electronic device may be a variety of types of devices, such as a mobile phone, a tablet computer, a server, etc. As shown in FIG. 25, the electronic device may include: a fourth processor 81 and a fourth memory 82. The fourth memory 82 is configured to store a program for executing the model training method provided in the embodiments shown in FIG. 16 by a corresponding electronic device, and the fourth processor 81 is configured to execute the program stored in the fourth memory 82.

A program includes one or more computer instructions, wherein the one or more computer instructions, when executed by the fourth processor 81, enable the following steps to be performed:

determining a processing resource corresponding to a model training service in response to a request for calling model training; and

performing the following steps with the processing resource: obtaining genetic samples, wherein the genetic samples correspond to sample mutation results, and an average number of genetic segments corresponding to each position in the genetic samples is less than or equal to a preset threshold; determining genetic features corresponding to the genetic samples and enhanced features corresponding to the genetic features; and performing learning and training based on reference genetic results, the genetic features and the enhanced features corresponding to the genetic samples to obtain a genetic testing model, wherein the genetic testing model is used for performing feature extraction operations on genetic data and performing testing operations on the genetic data based on extracted features.

Furthermore, the fourth processor 81 is also configured to perform all or part of the steps in the embodiments shown in FIG. 16.

The electronic device may further include a fourth communication interface 83 which is used by the electronic device for communicating with other devices or a communication network.

In addition, the embodiments of the present disclosure provide a computer storage medium configured to store computer software instructions used by an electronic device, which include a program for executing the model training method in the embodiments of the method shown in FIG. 16.

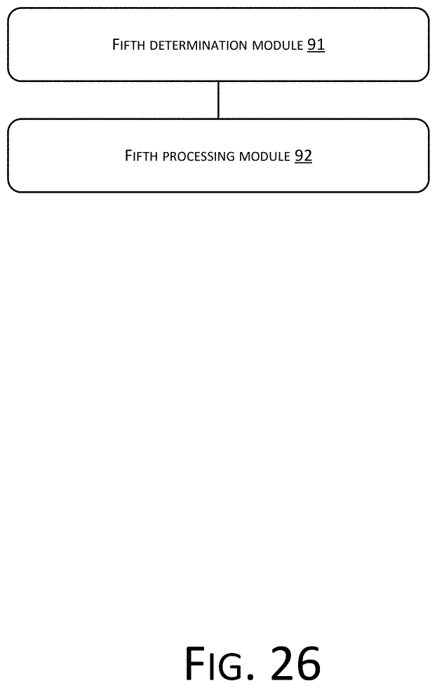

FIG. 26 is a schematic structural diagram of another genetic testing apparatus according to the embodiments of the present disclosure. Referring to FIG. 26, the embodiments of the present disclosure provide another genetic testing apparatus that can perform the genetic testing method shown in FIG. 17 described above. The genetic testing apparatus can include: a fifth determination module 91 and a fifth processing module 92, wherein:

the fifth determination module 91 configured to determine a processing resource corresponding to a model training service in response to a request for calling model training; and

the fifth processing module 92 configured to perform the following steps with the processing resource: obtaining genetic samples, wherein the genetic samples correspond to sample mutation results, and an average number of genetic segments corresponding to each position in the genetic samples is less than or equal to a preset threshold; determining genetic features corresponding to the genetic samples and enhanced features corresponding to the genetic features; and performing learning and training based on reference genetic results, the genetic features and the enhanced features corresponding to the genetic samples to obtain a genetic testing model, wherein the genetic testing model is used for performing feature extraction operations on genetic data and performing testing operations on the genetic data based on extracted features.

The apparatus shown in FIG. 26 can execute the method of the embodiments shown in FIG. 17. For parts that are not described in detail in the embodiments of the present disclosure may be referenced to related descriptions of the embodiments shown in FIG. 17. The implementation processes and technical effects of the technical solution are described in the embodiments shown in FIG. 17, and are not repeatedly described herein.

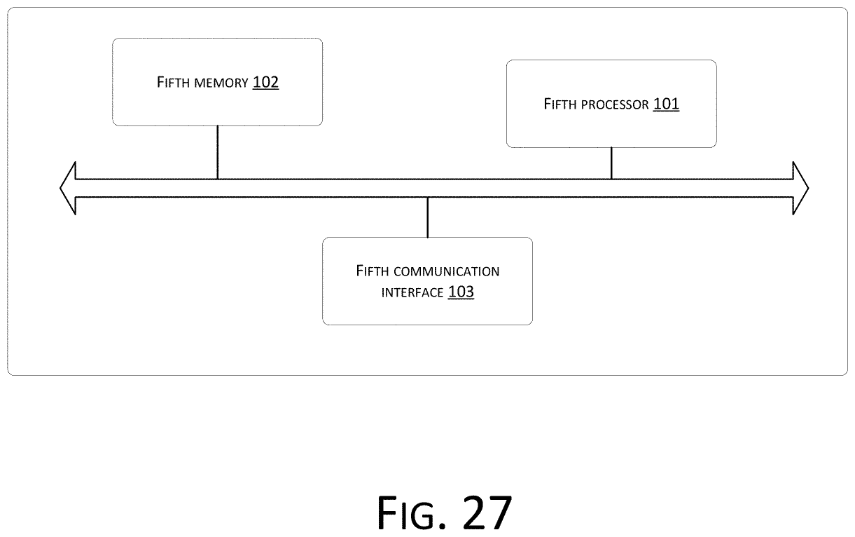

In a possible design, the structure of the model training apparatus shown in FIG. 26 may be implemented as an electronic device. The electronic device may be a variety of types of devices, such as a mobile phone, a tablet computer, a server, etc. As shown in FIG. 27, the electronic device may include: a fifth processor 101 and a fifth memory 102. The fifth memory 102 is configured to store a program for executing the model training method provided in the embodiments shown in FIG. 17 by a corresponding electronic device, and the fifth processor 101 is configured to execute the program stored in the fifth memory 102.

A program includes one or more computer instructions, wherein the one or more computer instructions, when executed by the fifth processor 101, enable the following steps to be performed:

determining a processing resource corresponding to a model training service in response to a request for calling model training; and

performing the following steps with the processing resource: obtaining genetic samples, wherein the genetic samples correspond to sample mutation results, and an average number of genetic segments corresponding to each position in the genetic samples is less than or equal to a preset threshold; determining genetic features corresponding to the genetic samples and enhanced features corresponding to the genetic features; and performing learning and training based on reference genetic results, the genetic features and the enhanced features corresponding to the genetic samples to obtain a genetic testing model, wherein the genetic testing model is used for performing feature extraction operations on genetic data and performing testing operations on the genetic data based on extracted features.

Further, the fifth processor 101 is also configured to perform all or part of the steps in the embodiments shown in FIG. 17.

The electronic device may further include a fifth communication interface 103 which is used by the electronic device to communicate with other devices or a communication network.

In addition, the embodiments of the present disclosure provide a computer storage medium configured to store computer software instructions used by an electronic device, which include a program for executing the genetic testing method in the embodiments of the method shown in FIG. 17.

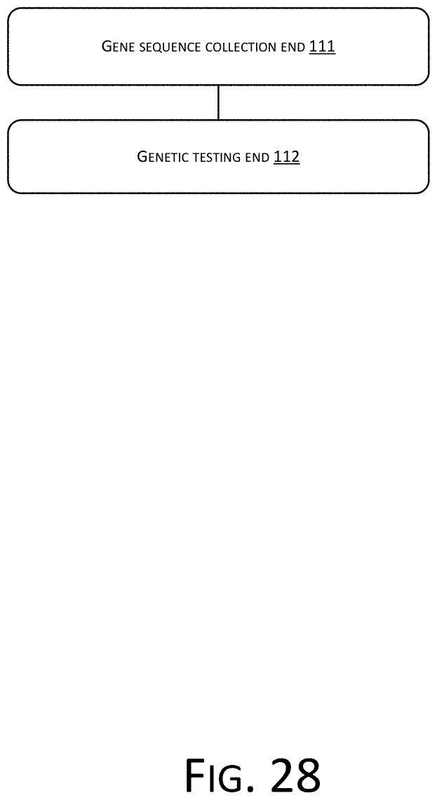

FIG. 28 is a schematic structural diagram of a genetic testing system according to the embodiments of the present disclosure. Referring to FIG. 28, the embodiments of the present disclosure provide a genetic testing system. The genetic testing system may include:

a gene sequence acquisition end 111 configured to obtain genetic data to be processed to be processed and transmit the genetic data to be processed to a genetic testing end, wherein an average number of genetic segments corresponding to each position in the genetic data to be processed is less than or equal to a preset threshold; and

the genetic testing end 112 in communication connection with the gene sequence acquisition end 111 and configured to determine a genetic testing model for analyzing and processing the genetic data to be processed, wherein the genetic testing model is trained to be used for performing a feature extraction operation on the genetic data to be processed and performing a testing operation on the genetic data to be processed based on extracted features; and analyzing and processing the genetic data to be processed using the genetic testing model to obtain a testing result.

The system shown in FIG. 28 may perform the methods of the embodiments shown in FIGS. 14 and 15. For parts that are not described in detail in the embodiments of the present disclosure may be referenced to related descriptions of the embodiments shown in FIGS. 14 and 15. The implementation processes and technical effects of the technical solution are described in the embodiments shown in FIGS. 14 and 15, and are not repeatedly described herein.

The foregoing apparatus embodiments are merely illustrative. Units described as separate parts may or may not be physically separate. Parts displayed as units may or may not be physical units, may be located in one place, or may be distributed over a plurality of network units. Some or all of the modules may be selected according to actual needs to achieve the purpose of the solutions of this embodiment. One of ordinary skill in the art can understand and implement them without making any inventive effort.

Through the above description of the embodiments, one skilled in the art will clearly understand that each embodiment can be implemented by adding a necessary general hardware platform, and apparently can also be implemented by a combination of hardware and software. With this understanding in mind, the essence of the above technical solutions or the portions that contribute to the existing technologies may be embodied in a form of a computer product. The present disclosure may adopt a form of a computer program product implemented on one or more computer-usable storage media (which includes, but are not limited to, disk storage, CD-ROM, optical storage, and the like) having computer-usable program codes embodied therein.

The present disclosure is described with reference to flowcharts and/or block diagrams of methods, apparatus (systems), and computer program products according to the embodiments of the present disclosure. It will be understood that each process and/or block of the flowcharts and/or block diagrams, and combinations of processes and/or blocks in the flowcharts and/or block diagrams, can be implemented by computer program instructions. These computer program instructions may be provided to a general purpose computer, a special purpose computer, an embedded processor, or a processor of other programmable device to produce a machine, to cause the instructions to generate an apparatus for implementing the functions specified in one or more processes of the flowcharts and/or one or more blocks of the block diagrams through the computer or the processor of other programmable device.

These computer program instructions may also be stored in a computer-readable storage device that can direct a computer or other programmable device to function in a particular manner, such that the instructions stored in the computer-readable storage device produce an article of manufacture including an instruction apparatus which implements the functions specified in one or more processes of the flowcharts and/or one or more blocks of the block diagrams.

These computer program instructions may also be loaded onto a computer or other programmable device to cause a series of operational steps to be performed on the computer or other programmable device so as to produce a computer implemented process, such that the instructions executed on the computer or other programmable device provide steps for implementing the functions specified in one or more processes of the flowcharts and/or one or more blocks of the block diagrams.

In a typical configuration, a computing device includes one or more processors (CPUs), input/output interface(s), network interface(s), and memory.

For example, each of the foregoing apparatus (such as the apparatuses shown in FIGS. 18, 20, 22, 24, and 26) and the foregoing system (such as the system shown in FIG. 28) may include one or more computing devices. In implementations, the foregoing apparatus or the foregoing system may include one or more processors, an input/output (I/O) interface, a network interface, and memory.

The memory may include a form of computer readable media such as a volatile memory, a random access memory (RAM) and/or a non-volatile memory, for example, a read-only memory (ROM) or a flash RAM. The memory is an example of a computer readable media.

The computer readable media may include a volatile or non-volatile type, a removable or non-removable media, which may achieve storage of information using any method or technology. The information may include a computer readable instruction, a data structure, a program module or other data. Examples of computer storage media include, but not limited to, phase-change memory (PRAM), static random access memory (SRAM), dynamic random access memory (DRAM), other types of random-access memory (RAM), read-only memory (ROM), electronically erasable programmable read-only memory (EEPROM), quick flash memory or other internal storage technology, compact disk read-only memory (CD-ROM), digital versatile disc (DVD) or other optical storage, magnetic cassette tape, magnetic disk storage or other magnetic storage devices, or any other non-transmission media, which may be used to store information that may be accessed by a computing device. As defined herein, the computer readable media does not include transitory media, such as modulated data signals and carrier waves.

Finally, it needs to be noted that: the above embodiments are only intended to illustrate the technical solutions of the present disclosure, but not to impose limitations thereon. Although the present disclosure has been described in detail with reference to the foregoing embodiments, one of ordinary skill in the art should understand that: the technical solutions described in the foregoing embodiments may be modified, or some technical features may be equivalently replaced. Such modifications or replacements do not depart from the spirit and scope of the corresponding technical solutions of the embodiments of the present disclosure.

What is claimed is:

1. A method implemented by one or more computing devices, the method comprising:
obtaining genetic data to be processed, wherein an average number of genetic segments corresponding to each position in the genetic data to be processed is less than or equal to a preset threshold;
inputting the genetic data to be processed into a feature generation network layer for performing a feature extraction operation to obtain genetic features corresponding to the genetic data to be processed and enhanced features corresponding to the genetic features; and
inputting the genetic data to be processed and the enhanced features into a genetic identification network layer for performing a genetic testing operation to obtain a testing result.

2. The method according to claim 1, wherein inputting the genetic data to be processed and the enhanced features into the genetic identification network layer for performing the genetic testing operation to obtain the testing result comprises:
performing genetic testing processing on the genetic data to be processed and the enhanced features using the genetic identification network layer to obtain testing reference information corresponding to the genetic data to be processed.

3. The method according to claim 2, wherein the testing reference information includes at least one of: 21-type genotype prediction information, zygotic prediction information, first allelic mutation length information, and second allelic mutation length information.

4. The method according to claim 2, wherein inputting the genetic data to be processed and the enhanced features into the genetic identification network layer for performing the genetic testing operation to obtain the testing result further comprises:
obtaining the testing result corresponding to the genetic data to be processed according to the testing reference information.

5. The method of claim 1, further comprising:
obtaining a standard data type corresponding to the genetic data to be processed;
inputting the genetic features to a data identification network layer for performing a data type identification operation to obtain a genetic data type;
determining a loss function used for the feature generation network layer based on the genetic data type and the standard data type; and
optimizing the feature generation network layer using the loss function to obtain an optimized feature generation network layer.

6. The method according to claim 5, wherein the feature generation network layer comprises a part of the data identification network layer, and optimizing the feature generation network layer using the loss function to obtain the optimized feature generation network layer comprises:
optimizing the data identification network layer based on the loss function to obtain an optimized data identification network layer; and
determining the optimized feature generation network layer based on the optimized data identification network layer.

7. One or more computer readable media storing executable instructions that, when executed by one or more processors, cause the one or more processors to perform acts comprising:
obtaining genetic samples, where the genetic samples correspond to sample mutation results, and an average number of genetic segments corresponding to each position in the genetic samples is less than or equal to a preset threshold;
determining genetic features corresponding to the genetic samples and enhanced features corresponding to the genetic features; and
performing learning and training based on reference genetic results, the genetic features, and the enhanced features corresponding to the genetic samples to obtain a genetic testing model, wherein the genetic testing model is configured to perform a feature extraction operation on genetic data and perform a testing operation on the genetic data based on extracted features.

8. The one or more computer readable media according to claim 7, wherein performing learning and training based on the reference genetic results, the genetic features, and the enhanced features corresponding to the genetic samples to obtain the genetic testing model comprises:
performing learning and training based on the genetic samples, the genetic features and the enhanced features to obtain a feature generation sub-model, wherein the feature generation sub-model is used for performing feature extraction and enhancing extracted genetic features;
performing learning and training based on the enhanced features and the reference genetic results corresponding to the genetic samples to obtain a variant identification model, wherein the variant identification model is used for testing genetic data based on feature information; and
generating the genetic testing model based on the feature generation sub-model and the variant identification model.

9. The one or more computer readable media according to claim 8, wherein after obtaining the feature generation sub-model, the acts further comprise:
performing learning and training based on the genetic features and the reference genetic results corresponding to the genetic samples to obtain a data identification model, wherein the data identification model is used for performing a variant identification operation on genetic data based on genetic features; and
optimizing the feature generation sub-model using the data identification model to obtain an optimized feature generation sub-model.

10. The one or more computer readable media according to claim 9, wherein the feature generation sub-model comprises a part of the data identification model, and optimizing the feature generation sub-model using the data identification model to obtain the optimized feature generation sub-model comprises:
obtaining a loss function used for optimizing the data identification model;
optimizing the data identification model based on the loss function to obtain an optimized data identification model; and
determining the optimized feature generation sub-model based on the optimized data identification model.

11. The one or more computer readable media according to claim 10, wherein obtaining the loss function used for optimizing the data identification model comprises:
analyzing and processing the genetic features using the data identification model to obtain predicted genetic results corresponding to the genetic features; and
determining the loss function used for optimizing the data identification model based on the genetic features, the predicted genetic results, and the reference genetic results.

12. The one or more computer readable media according to claim 8, wherein after obtaining the feature generation sub-model, the acts further comprise:
obtaining reference features for performing analysis processing on the enhanced features, wherein an average number of genetic segments corresponding to each position in the reference features is greater than the preset threshold;
performing learning and training based on the reference features and the enhanced features to obtain an adversarial discriminative model, wherein the adversarial discriminative model is configured to perform a discriminative operation on the genetic features; and
optimizing the feature generation sub-model using the adversarial discriminative model to obtain an optimized feature generation sub-model.

13. The one or more computer readable media according to claim 12, wherein optimizing the feature generation sub-model using the adversarial discriminative model to obtain the optimized feature generation sub-model comprises:
obtaining a judgment and identification result of analyzing and processing the enhanced features using the adversarial discriminative model; and
optimizing the feature generation sub-model based on the judgment and identification result to obtain the optimized feature generation sub-model.

14. The one or more computer readable media according to claim 7, the acts further comprising:
obtaining genetic data to be processed, wherein an average number of genetic segments corresponding to each position in the genetic data to be processed is less than or equal to a preset threshold;
determining the genetic testing model for analyzing and processing the genetic data to be processed; and
analyzing and processing the genetic data to be processed using the genetic testing model to obtain a testing result.

15. An apparatus comprising:
one or more processors; and
memory storing executable instructions that, when executed by the one or more processors, cause the one or more processors to perform acts comprising:
obtaining genetic data to be processed, wherein an average number of genetic segments corresponding to each position in the genetic data to be processed is less than or equal to a preset threshold;
inputting the genetic data to be processed into a feature generation network layer for performing a feature extraction operation to obtain genetic features corresponding to the genetic data to be processed and enhanced features corresponding to the genetic features; and
inputting the genetic data to be processed and the enhanced features into a genetic identification network layer for performing a genetic testing operation to obtain testing result.

16. The apparatus according to claim 15, wherein inputting the genetic data to be processed and the enhanced features into the genetic identification network layer for performing the genetic testing operation to obtain the testing result comprises:
performing genetic testing processing on the genetic data to be processed and the enhanced features using the genetic identification network layer to obtain testing reference information corresponding to the genetic data to be processed.

17. The apparatus according to claim 16, wherein the testing reference information includes at least one of: 21-type genotype prediction information, zygotic prediction information, first allelic mutation length information, and second allelic mutation length information.

18. The apparatus according to claim 16, wherein inputting the genetic data to be processed and the enhanced features into the genetic identification network layer for performing the genetic testing operation to obtain the testing result further comprises:
obtaining the testing result corresponding to the genetic data to be processed according to the testing reference information.

19. The apparatus of claim 15, the acts further comprising:
obtaining a standard data type corresponding to the genetic data to be processed;
inputting the genetic features to a data identification network layer for performing a data type identification operation to obtain a genetic data type;
determining a loss function used for the feature generation network layer based on the genetic data type and the standard data type; and
optimizing the feature generation network layer using the loss function to obtain an optimized feature generation network layer.

20. The apparatus according to claim 19, wherein the feature generation network layer comprises a part of the data identification network layer, and optimizing the feature generation network layer using the loss function to obtain the optimized feature generation network layer comprises:
optimizing the data identification network layer based on the loss function to obtain an optimized data identification network layer; and
determining the optimized feature generation network layer based on the optimized data identification network layer.
